(19) United States
(12) Patent Application Publication
(10) Pub. No.: US 2026/0234731 A1
(43) Pub. Date: Aug. 13, 2026

(54) HYDROXYMETHYLATION BIOMARKER LOCI IN THE DETECTION, MANAGEMENT, AND RECURRENCE PREDICTION OF CANCER, PARTICULARLY COLORECTAL CANCER

(71) Applicant: ClearNote Health, Inc., San Diego, CA (US)

(72) Inventors: Samuel LEVY, San Diego, CA (US); Gulfem Dilek GULER, San Diego, CA (US); Wayne VOLKMUTH, San Diego, CA (US); Ceyda CORUH, San Diego, CA (US); Yuhong NING, San Diego, CA (US)

(21) Appl. No.: 19/457,089

(22) Filed: Jan. 22, 2026

Related U.S. Application Data
(60) Provisional application No. 63/748,375, filed on Jan. 22, 2025.
(60) Provisional application No. 63/748,373, filed on Jan. 22, 2025.

(51) Int. Cl. C12Q 1/6886; C12Q 1/6809

(52) U.S. Cl. CPC C12Q 1/6886; C12Q 1/6809; C12Q 2600/106; C12Q 2600/118; C12Q 2600/154

(57) ABSTRACT

Disclosed herein are methods for identifying and using hydroxymethylation biomarker loci for predicting whether a cancer patient is likely to experience cancer recurrence after treatment with a cancer therapy. Also provided is a method for determining the probability that a cancer patient will or will not experience cancer recurrence following treatment with a cancer therapy, wherein the method involves hydroxymethylation analysis of a cell-free DNA (cfDNA) sample obtained from the patient using the aforementioned hydroxymethylation biomarker loci.

### CROSS-REFERENCE TO RELATED APPLICATIONS

This application claims priority under 35 U.S.C. § 119 (e) (1) to provisional U.S. patent application Ser. Nos. 63/748,373 and 63/748,375, filed Jan. 22, 2025. The disclosure of the aforementioned patent applications are incorporated by reference herein in their entireties.

### TECHNICAL FIELD

The present invention relates generally to the detection and treatment of colorectal cancer, and more particularly relates to the management of patients receiving a colorectal or other cancer treatment, e.g., surgical intervention. The invention additionally relates to methods for predicting the presence of cancer in a patient, the efficacy of treating a cancer patient, and the likelihood of recurrence post-treatment.

### BACKGROUND

Colorectal cancer is the third most common cancer in the United States, and is expected to claim more than 50,000 lives in 2023 (Morris et al., 2022). Diagnosis of colon or rectal cancer has been dropping over the last two decades in older adults due to several factors: an increase in the frequency of routine screening methods, which allows for removal of pre-cancerous polyps before they develop into cancer; improved treatments for colorectal cancer, resulting in better patient outcomes; and changes in lifestyle-related risk factors, e.g., cigarette smoking and obesity, which reduces the likelihood of cancer development. However, the incidence has been increasing among younger adults, and by 2030, colorectal cancer is predicted to be the major cause of cancer-related death for people between ages 20 and 49 in the United States (Cavallo et al. (2021)).

Approximately 33% of colorectal cancer patients will develop metastases throughout their cancer continuum, and their 5-year survival rate is about 15% (Morris et al., supra). Most metastatic colorectal cancer (mCRC) patients cannot be cured. However, a subset of mCRC patients with localized recurrence or liver- and/or lung-isolated metastatic disease can potentially be cured with surgery, with (or without) additional treatments (Clark and Sanoff, (2023). However, current means to select these patients with resectable metastatic disease are insufficient. Therefore, there is a dire need for predictive biomarkers that can identify these patients who will have a better prognosis following treatment, e.g., surgery, radiation therapy, chemotherapy, immunotherapy, or the like.

Current guidelines suggest that patients with metastatic colorectal cancer undergo surgical resection followed by adjuvant chemotherapy (Chiorean et al., 2020). While there are no known biomarkers that are used for mCRC patient stratification vis-a-vis the potential success of surgery, there are several predictive biomarkers that can be used to guide neo-adjuvant or adjuvant therapy selection. These include MSI-H/dMMR, RAS, BRAF, HER2, APC, CEA and NTRK (Crutcher et al., 2020; Clark and Sanoff, supra). Even though aforementioned biomarkers can help with patient stratification for drug selection, there is need for biomarkers that can predict patient outcome in response to surgical treatment using baseline signatures prior to a decision to perform surgery.

Lung cancer is the most common cancer worldwide and most cases are classified as either non-small cell lung cancer (NSCLC; 80%) or small cell lung cancer (SCLC; 14%) (American Cancer Society). It is also the leading cause of cancer-related deaths annually, estimated to claim 1.8 million lives globally in 2022 (Bray et al. 2024) and over 125,000 lives in the US in 2024 (SEER Cancer Stat Facts, National Cancer Institute, 2025). The prognosis and treatment options for lung cancer varies depending on multiple factors, such as the type and the stage of lung cancer, and whether the cancer has mutations in targetable genes. Among the treatment options available for NSCLC, immunotherapy has cemented its place in the management of advanced stage disease, achieving better patient outcomes in both first line setting and beyond, as mono or combination therapy, thereby resulting in FDA approvals in various regimens (Shields et al. 2021).

Cell-free DNA (cfDNA), averaging 167 bp in size, is composed of circulating DNA fragments derived from various origins including circulating tumor DNA (ctDNA), cell-free mitochondrial DNA, cell-free fetal DNA, donor-derived cell-free DNA and immune-derived cfDNA. Methylation analysis has demonstrated that megakaryocytes are major contributors (approximately 26%) of total cfDNA in healthy individuals (Moss et al., 2023). Megakaryocytes, originally identified as bone marrow-resident cells, produce platelets that are necessary for blood coagulation. However, research in the last few decades has showed that they may also reside in other tissues, such as lung, which might serve as reservoir for hematopoietic progenitors (Lefrançais et al. 2017). Additionally, tissue RNA-seq results of megakaryocytes obtained from mouse lung or bone marrow has revealed that lung-resident megakaryocytes express receptors that allows them to sense inflammation (Cunin and Nigrovic, 2019). Substantial contribution of megakaryocytes in the cfDNA pool identified via megakaryocyte-specific methylation patterns and their contribution to inflammation and immunity suggest that megakaryocytes may serve as biomarkers for disease detection and monitoring using plasma cfDNA.

To date, the gold standard for molecular profiling to guide clinical decision-making in cancer treatment, including CRC and lung cancer, remains tissue biopsy. However, this approach involves a test carried out at a single time point, fails to capture tumor heterogeneity, and is invasive, rendering it technically unfeasible in many cases. To circumvent these limitations, liquid biopsies, such as circulating tumor DNA (ctDNA) testing, circulating tumor cells (CTCs) and exosomes have been increasingly gaining attention as potential alternative tools to serve as prognostic, diagnostic, and monitoring biomarkers in cancer (Mauri et al., 2022). In the clinic, the most widely adopted liquid biopsy technique involves ctDNA-based platforms. Accumulating evidence shows that ctDNA can be highly prognostic, and clearance of ctDNA after surgery can be used as a biomarker that might help with therapy assessment and monitoring possible recurrence (Malla et al., 2022; Vidal et al., 2021; Reinert et al., 2019). Even though ctDNA is emerging as a powerful biomarker for various applications throughout the cancer continuum, the challenges for clinical implementation remain, such as standardization across different platforms. To this end, the Signatera test (Natera) for detecting molecular residual disease (MRD) has become commercially available as a personalized ctDNA test to detect cancer recurrence; however, that test requires tumor tissue signatures.

There is, therefore, a need for an improved method to predict which cancer patients, particularly colorectal and lung cancer patients, would likely fare better after treatment, e.g., after a surgical intervention (as may be measured, for instance, by the statistical likelihood of recurrence over a given period of time), including metastatic colorectal cancer patients. Ideally, the method would not require tissue biopsy but rather rely on biomarkers that can inform on tumor biology without a tissue biopsy.

There is also a need for biomarkers useful in diagnosis or treatment assessment following treatment. Currently, imaging is the main modality that is used not only for diagnosis but also for treatment assessment. There is a blood test that assesses serum carcinoembryonic antigen (CEA) levels periodically that is widely used for monitoring treatment response; however, despite the correlation between rising CEA levels and disease progression for many cases, CEA levels alone are insufficient due to suboptimal performance, exhibiting a wide range of sensitivity and specificity values (Nicholson et al., 2015). As a result, it is generally recommended that oncologists obtain a confirmatory radiologic response and consider the histology of the tumor prior to making decisions about changing therapeutic strategy (Clark and Sanoff, 2023, supra). ASCO-resource stratified guidelines strongly recommend either x-ray or contrast-enhanced CT scans as means for determining cancer stage (Chiorean et al., 2020, supra). These methods are also problematic, as, for example, multiple radiological evaluations over a relatively short course of treatment are not recommended, as such frequent evaluations could expose the patient to excessive radiation doses.

Accordingly, development of minimally invasive liquid biomarkers that can provide information about tumor biology and likely treatment result is needed to predict patient outcome in cancer patients such as colorectal cancer and lung cancer patients, including metastatic colorectal cancer patients, after surgery or another treatment modality.

### SUMMARY OF THE INVENTION

Tumor and normal cell DNA is released into the bloodstream, and a cell-free DNA (cfDNA) sample extracted therefrom can be analyzed with respect to genetic and epigenetic signatures. Epigenetic signatures include, by way of example, DNA methylation and DNA hydroxymethylation, with 5hmC profile (or “hydroxymethylome” or “hydroxymethylation signature”) of particular interest herein.

The present invention is directed, in part, to a method for identifying a set of hydroxymethylation biomarkers that, optionally in combination with one or more other types of biomarkers, features, and/or patient-specific characteristics, correlates with the likelihood that a patient has cancer, e.g., a particular type of cancer such as lung cancer or colorectal cancer, and/or that such cancer patients will remain recurrence-free during a follow-up time period after the patient is treated with a specific cancer therapy. A “biomarker” as that term is used herein refers to a characteristic that can be measured as an indicator of normal biological processes, pathogenic processes, or responses to an exposure or intervention, including therapeutic interventions.

In a first embodiment, the invention provides a method for identifying differentially hydroxymethylated cytosine sites useful as hydroxymethylation biomarkers in predicting a probability that a cancer patient who has undergone treatment for the cancer will experience recurrence within a follow-up time period after the treatment, wherein the method comprises:
(a) obtaining an initial cfDNA sample from each of a plurality of cancer patients prior to beginning treatment with the cancer therapy;
(b) determining a baseline count T₀ of hydroxymethylated cytosine sites in CPM at each of a plurality of candidate hydroxymethylation biomarker loci in the initial cfDNA samples;
(c) treating the patients with the cancer therapy;
(d) at the conclusion of the follow-up time period, confirming recurrence or nonrecurrence of cancer to identify a first population of cancer recurrent patients and a second population of cancer nonrecurrent patients;
(e) determining a correlation between the T₀ count at the hydroxymethylated cytosine sites and recurrence or nonrecurrence of cancer in the patients; and
(f) selecting as the hydroxymethylation biomarkers the hydroxymethylated cytosine sites exhibiting a sufficient correlation with recurrence or nonrecurrence.

In another embodiment, the invention provides a method for identifying differentially hydroxymethylated cytosine sites in cfDNA useful as hydroxymethylation biomarkers in predicting a probability that a cancer patient who has undergone treatment for the cancer will experience recurrence within a follow-up time period after the treatment, wherein the method comprises:
(a) obtaining an initial cfDNA sample from each of a plurality of cancer patients prior to beginning treatment with the cancer therapy;
(b) determining a baseline count T₀ of hydroxymethylated cytosine sites in CPM at each of a plurality of candidate hydroxymethylation biomarker loci in the initial cfDNA samples;
(c) treating the patients with the cancer therapy;
(d) at the conclusion of the follow-up time period, (i) confirming recurrence or nonrecurrence of cancer to identify a first population of cancer recurrent patients and a second population of cancer nonrecurrent patients and (ii) obtaining a subsequent cfDNA sample from each of the patients;
(e) determining a subsequent count T_{S} at each of the candidate hydroxymethylation biomarker loci in the subsequent cfDNA samples; and
(f) adopting as hydroxymethylation biomarker loci those candidate hydroxymethylation biomarker loci exhibiting a threshold p-value of less than 0.05 and a difference ξ of at least 1.5, wherein

$$ζ = ( {T}_{S} - {T}_{0} ) / {T}_{0} .$$

In one aspect of the aforementioned embodiments, the cancer is colorectal cancer and the cancer therapy is a colorectal cancer therapy.

In another aspect, the cancer is lung cancer and the cancer therapy is a lung cancer therapy.

In another aspect, the cancer is ovarian cancer and the cancer therapy is an ovarian cancer therapy.

In another aspect, the cancer is breast cancer and the cancer therapy is a breast cancer therapy.

In another aspect, the cancer is pancreatic cancer and the cancer therapy is a pancreatic cancer therapy.

Accordingly, in another aspect, the invention provides a method for identifying differentially hydroxymethylated sites useful as hydroxymethylation biomarkers in predicting a probability that a colorectal cancer patient who has been treated will experience recurrence within a follow-up time period after treatment with a colorectal cancer therapy, wherein the method comprises:
(a) obtaining an initial cfDNA sample from each of a plurality of colorectal cancer patients prior to beginning treatment with the colorectal cancer therapy;
(b) determining a baseline count T₀ of hydroxymethylated cytosine sites in CPM at each of a plurality of candidate hydroxymethylation biomarker loci in the initial cfDNA samples;
(c) treating the patients with the colorectal cancer therapy;
(d) at the conclusion of the follow-up time period, confirming recurrence or nonrecurrence of colorectal cancer to identify a first population of cancer recurrent patients and a second population of cancer nonrecurrent patients;
(e) determining a correlation between the T₀ count at the hydroxymethylated cytosine sites and recurrence or nonrecurrence of cancer in the patients; and
(f) selecting as the hydroxymethylation biomarkers the hydroxymethylated cytosine sites exhibiting a sufficient correlation with recurrence or nonrecurrence.

In another aspect, the invention provides a method for identifying differentially hydroxymethylated sites useful as hydroxymethylation biomarkers in predicting a probability that a lung cancer patient who has been treated will experience recurrence within a follow-up time period after treatment with a lung cancer therapy, wherein the method comprises:
(a) obtaining an initial cfDNA sample from each of a plurality of lung cancer patients prior to beginning treatment with the lung cancer therapy;
(b) determining a baseline count T₀ of hydroxymethylated cytosine sites in CPM at each of a plurality of candidate hydroxymethylation biomarker loci in the initial cfDNA samples;
(c) treating the patients with the lung cancer therapy;
(d) at the conclusion of the follow-up time period, confirming recurrence or nonrecurrence of lung cancer to identify a first population of cancer recurrent patients and a second population of cancer nonrecurrent patients;
(e) determining a correlation between the T₀ count at the hydroxymethylated cytosine sites and recurrence or nonrecurrence of cancer in the patients; and
(f) selecting as the hydroxymethylation biomarkers the hydroxymethylated cytosine sites exhibiting a sufficient correlation with recurrence or nonrecurrence.

In a further aspect, the invention provides a method for identifying differentially hydroxymethylated cytosine sites in cfDNA useful as hydroxymethylation biomarkers in predicting a probability that a colorectal cancer patient who has undergone treatment for the colorectal cancer will experience recurrence within a follow-up time period after the treatment, wherein the method comprises:
(a) obtaining an initial cfDNA sample from each of a plurality of colorectal cancer patients prior to beginning treatment with the cancer therapy;
(b) determining a baseline count T₀ of hydroxymethylated cytosine sites in CPM at each of a plurality of candidate hydroxymethylation biomarker loci in the initial cfDNA samples;
(c) treating the patients with the colorectal cancer therapy;
(d) at the conclusion of the follow-up time period, (i) confirming recurrence or nonrecurrence of cancer to identify a first population of cancer recurrent patients and a second population of cancer nonrecurrent patients and (ii) obtaining a subsequent cfDNA sample from each of the patients;
(e) determining a subsequent count T_{S} at each of the candidate hydroxymethylation biomarker loci in the subsequent cfDNA samples; and
(f) adopting as hydroxymethylation biomarker loci those candidate hydroxymethylation biomarker loci exhibiting a threshold p-value of less than 0.05 and a difference (of at least 1.5, wherein

$$ζ = ( {T}_{S} - {T}_{0} ) / {T}_{0} .$$

In a further aspect, the invention provides a method for identifying differentially hydroxymethylated cytosine sites in cfDNA useful as hydroxymethylation biomarkers in predicting a probability that a lung cancer patient who has undergone treatment for the lung cancer will experience recurrence within a follow-up time period after the treatment, wherein the method comprises:
(a) obtaining an initial cfDNA sample from each of a plurality of lung cancer patients prior to beginning treatment with the lung cancer therapy;
(b) determining a baseline count T₀ of hydroxymethylated cytosine sites in CPM at each of a plurality of candidate hydroxymethylation biomarker loci in the initial cfDNA samples;
(c) treating the patients with the lung cancer therapy;
(d) at the conclusion of the follow-up time period, (i) confirming recurrence or nonrecurrence of cancer to identify a first population of cancer recurrent patients and a second population of cancer nonrecurrent patients and (ii) obtaining a subsequent cfDNA sample from each of the patients;
(e) determining a subsequent count T_{S} at each of the candidate hydroxymethylation biomarker loci in the subsequent cfDNA samples; and
(f) adopting as hydroxymethylation biomarker loci those candidate hydroxymethylation biomarker loci exhibiting a threshold p-value of less than 0.05 and a difference ξ of at least 1.5, wherein

$$ζ = ( {T}_{S} - {T}_{0} ) / {T}_{0} .$$

In some aspects, the follow-up time period is one month, three months, six months, nine months, one year, eighteen months, two years, three years, or five years.

In some aspects, the cancer therapy, e.g., colorectal cancer therapy, lung cancer therapy, or other cancer therapy, comprises surgical resection of a cancerous lesion. In other aspects, the cancer therapy comprises surgical resection of a cancerous lesion followed by adjuvant chemotherapy.

The cancer therapy may also comprise, in some embodiments, radiation therapy, targeted therapy, immunotherapy and/or chemotherapy as neo-adjuvant or adjuvant therapy, in addition to or in lieu of a surgical treatment.

In another embodiment, the invention provides a method for determining the probability that a cancer patient, e.g., a colorectal cancer patient or lung cancer patient, will or will not experience cancer recurrence within a follow-up time period after treatment with a cancer therapy, wherein the method comprises, prior to treatment:
(a) obtaining a cfDNA sample from the patient, enriching for hydroxymethylated DNA in the sample, amplifying the hydroxymethylated DNA, and sequencing the amplified hydroxymethylated DNA in a manner that identifies 5hmC-containing fragments in the DNA;
(b) determining a hydroxymethylation signature for the patient by identifying the extent of hydroxymethylation in the 5hmC-containing fragments at each of the hydroxymethylation biomarker loci adopted according to the method of the above embodiment;
(c) using the hydroxymethylation signature, calculating a probability score representing the probability that the cancer patient, e.g., the colorectal cancer patient or lung cancer patient, will or will not experience cancer recurrence within the follow-up time period.

As with the previous embodiments, the follow-up time period is three months, six months, nine months, one year, eighteen months, two years, three years, or five years.

In some aspects, the cancer therapy comprises surgical resection of a cancerous lesion. In other aspects, the cancer therapy comprises surgical resection of a cancerous lesion followed by adjuvant chemotherapy.

The cancer therapy, i.e., the treatment modality selected to treat colorectal cancer, lung cancer, or another cancer, may also comprise, in some embodiments, radiation therapy, targeted therapy, immunotherapy and/or chemotherapy as neo-adjuvant or adjuvant therapy, in addition to or in lieu of surgical treatment.

In some aspects, the cancer patient is a colorectal cancer patient who has metastatic colorectal cancer. In other aspects, the cancer patient is a lung cancer patient who has SCLC or NSCLC.

In some aspects, the probability score represents the probability that the colorectal cancer patient, lung cancer patient, or other cancer patient will experience cancer recurrence within the follow-up time period.

In other aspects, the probability score represents the probability that the colorectal cancer patient, lung cancer patient, or other cancer patient will not experience cancer recurrence within the follow-up time period.

In some aspects, step (c) comprises calculating the probability score using the hydroxymethylation signature and at least one additional feature type, where the additional feature type may comprise a clinical feature and/or at least one of cfDNA concentration in the cfDNA sample, 5hmC-containing fragment count in a selected genomic location, 5hmC-containing fragment size, copy number variation in the cfDNA, estimated tumor fraction, fragment end analysis, nucleosome positioning, and methylation data.

By “responding positively to colorectal cancer therapy” or “responding positively to lung cancer therapy,” and “responding to colorectal cancer therapy” or “responding to lung cancer therapy” is meant that the cancer patient treated with the cancer therapy exhibits a Complete Response (CR) Partial Response (PR), Stable Disease (SD) or Progressive Disease (PD) for a period of at least six months, as defined in the RECIST 1.1 guidelines as set forth in Eisenhauer et al. (2009), the disclosure of which is incorporated by reference herein. Colorectal or other cancer patients who exhibit Progressive Disease (PD), as that term is also defined in the RECIST 1.1 guidelines, are deemed nonresponders to treatment with the selected colorectal or other cancer therapy. Also see Spindler et al. (2023).

In some embodiments, the plurality of the hydroxymethylation biomarkers are located within gene bodies associated with genes and gene sets involved in the tumor progression-relevant pathways discussed in the examples and illustrated in FIG. 3. These gene sets include epithelial mesenchymal transition (EMT), G2M checkpoint, E2F targets, mTORC1 signaling, MYC targets, DNA repair, P53, and oxidative phosphorylation (OXPHO). In some embodiments, the plurality of the hydroxymethylation biomarker loci are within gene bodies associated with beta-catenin, MYC (c-myc), OXPHO, and G2M checkpoint-involving genes and gene sets. It will be appreciated that the hydroxymethylation biomarkers may be located within gene bodies associated with other genes and gene sets as well, as may be determined using the techniques described herein, optionally in combination with other techniques currently known to or hereinafter discovered by those in the field.

If a cancer therapy, e.g., a colorectal cancer therapy or lung cancer therapy, is determined to be potentially useful in treating the patient, i.e., because the calculated probability score exceeds a predefined threshold, the patient can undergo treatment with the selected therapy. If, on the other hand, the calculated probability score indicates that the patient is unlikely to respond to treatment with a particular cancer therapy, a decision to pursue a different course of action can be made. The patient is thereby spared unnecessary treatment and loss of valuable time during the course of the disease.

As may be deduced from the above, the selected loci that serve as hydroxymethylation biomarkers herein comprise loci selected for their relevance to responsiveness to treatment with a cancer therapy such as surgical resection of a cancerous tumor. By “relevance” is meant that a hydroxymethylation biomarker locus, alone or in combination with one or more other features, tends to exhibit an increase or decrease in hydroxymethylation in a manner that correlates with the likely treatment responsiveness of the cancer patient, with regard to, for example, tumor size, stage, invasiveness, grade, and the like. In general, relevance can be determined by assessing the correlation between a potential biomarker or biomarker set and the likelihood that the cancer patient will respond to treatment with a particular cancer therapy. That correlation, as briefly alluded to above, generally, although not necessarily, involves (a) a fold change of at least 1.5 between the hydroxymethylation level at a particular locus in a responding subject and the hydroxymethylation level at the same locus for a nonresponding subject, and (b) differential hydroxymethylation between responders and nonresponders with a p-value of less than 0.05 as determined by the Wilcoxon rank-sum test (Mann et al., 1947).

The methods of the present invention provide an improvement over currently available methods of evaluating the likelihood that a subject with cancer is likely to respond to treatment with a cancer therapy, insofar as those methods are largely based on clinical symptoms and radiographic evaluation, or on biomarkers that are not sufficiently predictive. The term “cancer” as used herein, e.g., with regard to “a cancer patient” or “a cancer therapy,” is intended to include any type of cancer involving the presence of a solid tumor, wherein the cancer may be colorectal cancer or lung cancer as discussed above.

In additional embodiments, the invention provides methods for using the selected hydroxymethylation biomarker loci in the following contexts:

Determining a course of treatment for a subject with colorectal cancer, lung cancer, or another cancer;

Determining whether a therapy used to treat a subject with colorectal cancer, lung cancer, or another cancer should be discontinued;

Determining an alternative course of treatment after an initial cancer therapy is discontinued; and

Reducing the risk that a subject with cancer who is unlikely to positively respond to a treatment with a particular cancer therapy will receive that therapy.

The invention also provides a method for ascertaining whether a cancer patient, e.g., a colorectal cancer patient, a lung cancer patient, or other cancer patient, is responding to treatment with a particular cancer therapy by calculating a 5hmC molecular response score MR_{5hmc} from analysis of 5hmC levels at selected 5hmC biomarker loci, with a positive value generally indicating that the patient will likely respond to the therapy and a negative value generally indicating that the patient is a likely nonresponder.

By “responding” to a cancer therapy as the term is used herein is meant that the calculated probability score exceeds a predefined threshold and so indicates that the patient is unlikely to experience cancer recurrence within the follow-up time period.

In another embodiment, the invention provides a method for determining whether a cancer patient, e.g., a colorectal cancer patient, lung cancer patient, or other cancer patient, is responding to a selected cancer therapy, the method comprising:
(a) in a cfDNA sample obtained from the patient, determining a baseline count T₀ of hydroxymethylated cytosine sites at each of the hydroxymethylation biomarker loci selected according to the above method for identifying differentially hydroxymethylated sites suitable as hydroxymethylation biomarkers in evaluating whether a cancer patient, e.g., a colorectal cancer patient, a lung cancer patient, or other cancer patient is a likely responder or a nonresponder to a selected cancer therapy;
(b) in a later cfDNA sample obtained from the patient, determining a later count T_{Q} at each of the selected hydroxymethylation biomarker loci after beginning the therapy;
(c) calculating a value for

$${log}_{2} ( {T}_{Q} / {T}_{0} )$$

at each of the selected hydroxymethylated biomarker loci, and identifying the calculated values as xᵢ at each locus i or yⱼ at each locus j, wherein the xᵢ and yⱼ are positively and negatively correlated with treatment response, respectively;
(d) calculating a 5hmC molecular response score (MR_{5hmc}) for the patient using the equation

$${MR}_{5 hmC} = {μ}_{x} - {μ}_{y}$$

wherein μₓ is the mean of the xᵢ over i loci and μ_{y} is the mean of the yⱼ over j loci; and
(e) determining that the patient is responding to the therapy when the 5hmC molecular response score is positive.

In another embodiment of the invention, a method is provided for determining that the likelihood that a patient has cancer is higher than the likelihood that the patient does not have cancer, wherein the method relies on the discovery that megakaryocyte-derived cfDNA exhibits reduced 5hmC representation in cancer patients relative to that seen in non-cancer patients. The method of this embodiment thus comprises obtaining a cfDNA sample from a patient, evaluating 5hmC levels in megakaryocyte-derived cfDNA in the sample, comparing the megakaryocyte 5hmC levels seen with that observed for a cancer patient or a non-cancer patient, and determining that the patient is more or less likely to have cancer based on the result. As described in the examples, reduced 5hmC levels were seen in all of the C8 gene sets associated with megakaryocytes.

In a further embodiment of the invention, a method is provided for determining the likelihood that a cancer patient will relapse within two years of undergoing a cancer treatment, wherein the method relies on the discovery that elevated 5hmC levels are observed over the Hallmark WNT/β-catenin pathway genes in patients who relapsed within two years of having had cancer treatment, particularly a surgical intervention.

In still a further embodiment, a method is provided for determining the likelihood that a colorectal cancer patient will exhibit recurrence within two years of treatment, or whether the patient is more or less likely to exhibit recurrence, wherein the method comprises assessing gene body 5hmC levels in a cfDNA sample taken from the patient, particularly transcription factor 5hmC levels such as CDX2 5hmC levels, and determining that the cfDNA has a higher fraction of tumor-generated cfDNA when increased 5hmC is seen at the transcription factor target regions. A higher fraction of tumor-generated cfDNA correlates with an increased likelihood that the colorectal cancer patient will exhibit recurrence within two years of treatment.

### BRIEF DESCRIPTION OF THE DRAWINGS

The file of this patent contains at least one drawing executed in color. Copies of this patent with color drawings will be provided by the Patent and Trademark Office upon request and payment of the necessary fee.

FIG. 1 a PCA plot using gene body 5hmC profiles (CPM) of non-relapsed (No_relapse) patients, defined as patients who have not relapsed within 2 years of surgery, and relapsed patients, defines as patients who relapsed within 2 years of surgery. To identify differential 5hmC marks (DhMRs), edgeR analysis was performed between relapsed versus non-relapsed patients. 1,348 genes were identified with differential 5hmC levels (p<0.05) in relapsed patients compared to non-relapsed patients.

FIG. 2 provides the results of edgeR analysis showing genes with differential 5hmC gene body counts between relapsed and non-relapsed patients, as also described in part (B) of Example 1.

FIG. 3 provides the results of gene set enrichment analysis (GSEA) performed using the Hallmark and C6 databases to identify biological processes that can be distinguished by comparing the cfDNA 5hmC profiles of relapsed and non-relapsed patients; see part (B) of Example 1.

FIG. 4 provides boxplots showing mean baseline 5hmC levels (CPM) over beta-catenin, oxidative phosphorylation (OXPHO), and G2M checkpoint gene sets between relapsed- and non-relapsed patients; see part (B) of Example 1.

FIG. 5 provides AUROC curves calculated using mean baseline 5hmC levels (CPM) over beta-catenin, oxidative phosphorylation (OXPHO), and G2M checkpoint gene sets between relapsed (and non-relapsed patients; see part (B) of Example 1.

FIG. 6 is a receiver-operating characteristic curve from predictive modeling using a regularized regression model (elastic net) on the training dataset with 294 colorectal cancer and 588 non-cancer cfDNA across 10-fold validation, as explained in Example 2.

FIG. 7 shows the cancer prediction score of CRC and non-cancer samples as determined in Example 2.

FIG. 8 is a receiver-operating characteristic curve of 69 colorectal cancer and 70 non-cancer cfDNA samples, obtained as described in Example 2.

FIG. 9 is a pie chart indicating the percentage of patients with different types of cancers, as described in Example 3.

FIG. 10 is a pie chart indicating the cancer stage distribution of patient samples evaluated as also described in Example 3.

FIG. 11 illustrates the age (A) and BMI (B) distribution of all cancer and non-cancer samples (Wilcoxon test) as described in Example 3.

FIG. 12 illustrates the age and BMI distributions of breast (A), colorectal (B), ovarian (C), and pancreatic (D) cancer cohorts along with their matched non-cancer cohorts (Wilcoxon test) as described in Example 3.

FIG. 13 is an MA plot of differentially hydroxymethylated genes (DhMGs) in cfDNA, comparing all cancer samples with non-cancer samples, as explained in Example 3. The upper (red) and lower (blue) dots indicate increased or decreased 5hmC density in cancer compared to non-cancer samples (FDR≤0.05).

FIG. 14 shows the GSEA C8 normalized enrichment scores of the top positive and negative representative pathways in cancers relative to non-cancers.

FIG. 15 provides the normalized enrichment scores (NES) of megakaryocyte gene sets between cancer and non-cancer samples. Each data point in a boxplot represents a megakaryocyte gene set (C8) with differential enrichment at FDR<0.05.

FIG. 16 shows the age and sex distribution of CRC and non-cancer samples evaluated as described in Example 4.

FIG. 17 is an MA plot of DhMGs in cfDNA, comparing CRC samples versus non-cancer samples (FDR<0.05). Upper (red) and lower (blue) dots indicate increased or decreased 5hmC density in CRC compared to non-cancer samples, respectively.

FIG. 18 shows the result of gene set enrichment analysis using C8 cell-type signature genes, as described in Example 4. 5hmC levels in genes related to megakaryocytes were found to be significantly lower in CRC patients compared to non-cancer patient samples, potentially suggesting a higher proportion of cfDNA derived from colon-tissue specific genes in cancer samples.

FIG. 19 is a boxplot showing cumulative 5hmC levels (sum of FPKM) over 71 genes that are common between the colon crypt-specific gene set and genes with significantly increased 5hmC representation in CRC relative to non-cancer samples.

FIG. 20 provides the MA plot from the edgeR analysis described in Example 5, showing genes with differential 5hmC gene body counts in CRC patients compared to the non-cancer control.

FIG. 21 shows a Hallmark pathway analysis that reveals increased 5hmC gene body levels across pathways associated with enhanced cell proliferation, metabolic activity, stress responses, oncogenic signaling, and cell-cycle activation, consistent with progressive tumor biology (Example 5).

FIG. 22 is a bar plot showing the top 20 C8 gene sets with differential gene body 5hmC counts between CRC samples and non-cancer controls. NES>0 indicates differential 5hmC enrichment in CRC samples while NES<0 indicates enrichment in non-cancer controls (FDR<0.05) (Example 5).

FIG. 23 is a bar plot showing the top 20 C6 gene sets with differential gene body 5hmC counts between CRC samples and non-cancer controls. NES>0 indicates differential 5hmC enrichment in CRC samples while NES<0 indicates enrichment in non-cancer controls (FDR<0.05) (Example 5).

FIG. 24 schematically illustrates the study design underlying the experimental work described in Example 6.

FIG. 25 shows the age distribution between NSCLC and non-cancer samples (Wilcoxon test), and the sex distribution of NSCLC and non-cancer samples was as follows: lung cancer (CRPR plus PD), 18 (58.1%) female, 13 (41.9%) male; non-cancer, 37 (59.7%) female, and 25 (40.3%) male.

FIG. 26 provides the results of GSEA using 5hmC counts over cell-type signature genes (C8) comparing NSCLC relative to non-cancer samples, where red (upper right) and blue (lower left) show elevated and reduced 5hmC representation in NSCLC relative to non-cancer, respectively.

As described in Example 6, gene body 5hmC profiles were also examined at the time of response compared to anti-PD1 treatment start which revealed differential profiles for anti-PD-1 responders relative to non-responders after initiation of treatment. FIG. 27 and FIG. 28 show 5hmC counts over genes in cell type signatures compared between time of response (T_{R}) and baseline (T₀), revealing several pathways associated with activation of immune response in responding patients and epithelial and stromal cell types in non-responders in a larger set (FIG. 27) and a smaller set (FIG. 28).

FIG. 29 is a boxplot showing the mean change in 5hmC levels (FPKM) over lung megakaryocyte gene set between time of response (TR) and baseline (T0) for responders (CR+PR (18)) and non-responders (PD, 13), as described in Example 6.

FIG. 30 is a boxplot showing the mean change in 5hmC levels (FPKM) between time of response (T_{R}) and baseline (T₀) for (CR+PR (23)) and non-responders (PD, 18), as also described in Example 6. It can be seen that the change seen in FIG. 20 became even more significant with the increased sample size for both cohorts.

FIG. 31 shows that the median change in megakaryocyte 5hmC levels after immunotherapy was positive in responders (CR+PR) and negative in non-responders (PD), suggesting increased tumor load in non-responders.

FIG. 32 shows that the patients evaluated who exhibited increased 5hmC levels over megakaryocytes had significant better overall survival compared to patients with decreased 5hmC levels after treatment.

FIG. 33 is a table that provides the clinical characteristics of the cancer and non-cancer cohorts evaluated as described in Example 7.

FIG. 34 is a pie chart indicating the cancer stage distribution of samples collected in Streck tubes, as described in Example 7.

FIG. 35 shows independent validation of a data set comprised of 70 non-cancer (EDTA) and 75 CRC (EDTA) samples, showing robust discrimination between colorectal cancer and non-cancer samples, with an area under the curve (AUC) of 0.939. See Example 7.

FIG. 36, similarly, provides an ROC curve obtained using a colorectal cancer detection model generated using well-balanced CRC (n=69; 40.6% female) and non-cancer (n=70; 42.9% female) cohorts comprised of patients whose blood was collected in EDTA tubes. The ROC curve resulted in an AUC of 0.999 using 10-fold outer CV for the EDTA data set.

FIG. 37 is a boxplot of Stage IV CRC patients' prediction scores between relapsed patients and non-relapsed patients, as described in Example 8.

FIG. 38 shows the correlation of pre-surgery cancer detection score with decreased overall survival. Kaplan-Meier survival curves stratified by pre-surgery cancer detection status demonstrate improved overall survival in patients without detectable cancer.

FIG. 39 is a boxplot showing significantly (p=0.013) elevated 5hmC levels over the Hallmark WNT/β-catenin pathway genes in patients who relapsed within two years post-surgery compared to the patients who did not relapse, with no evidence of disease, for at least two years after surgery.

FIG. 40 is a Kaplan-Meier plot demonstrating the prognostic value of cfDNA 5hmC over WNT/β-catenin pathway genes through overall survival, indicating its potential role in predicting patient outcomes (p=0.02). Stage IV samples were stratified into high and low groups based on Wnt/β-catenin 5hmC levels above or below the median, respectively. Samples with 10-year follow-up data were included in the plot.

FIG. 41 provides scatter plots showing correlations between CDX2 mean coverage (left), central coverage (middle), and amplitude (right) against tumor fractions estimated by ichorCNA, as described in Example 9.

FIG. 42 provides boxplots showing correlations between CDX2 mean coverage (left), central coverage (middle), and amplitude (right) over non-cancer samples and CRC samples grouped by having low or high ctDNA fractions as estimated by ichorCNA, as described in Example 9.

FIG. 43 provides scatter plots showing correlations between CDX2 mean coverage (left), central coverage (middle), and amplitude (right) against increased CDX2 5hmC levels, as described in Example 9.

FIG. 44 provides scatter plots showing correlations between CDX2 mean coverage (left), central coverage (middle), and amplitude (right) against increased 5hmC levels over CDX2 target binding sites, as described in Example 9.

FIG. 45 provides AUROC curve plots obtained in carrying out GMLNET prediction of CRC using cfDNA nucleosome accessibility, and showing the performance of the training set using 10-fold CV (left) and the test set (B), as described in Example 9.

### DETAILED DESCRIPTION OF THE INVENTION

### 1. Terminology

Unless defined otherwise, all technical and scientific terms used herein have the meaning commonly understood by one of ordinary skill in the art to which the invention pertains. Specific terminology of particular importance to the description of the present invention is defined below. Other relevant terminology is defined in International Patent Publication No. WO 2017/176630 to Quake et al. for “Noninvasive Diagnostics by Sequencing 5-Hydroxymethylated Cell-Free DNA.” The aforementioned patent publication as well as all other patent documents and publications referred to herein are expressly incorporated by reference.

In this specification and the appended claims, the singular forms “a,” “an” and “the” include plural referents unless the context clearly dictates otherwise. Thus, for example, “an adapter” refers not only to a single adapter but also to two or more adapters that may be the same or different, “a template molecule” refers to a single template molecule as well as a plurality of template molecules, and the like.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention. Accordingly, the terms defined immediately below are more fully defined by reference to the specification as a whole.

The term “sample” as used herein relates to a material or mixture of materials, typically, although not necessarily, in liquid form, containing one or more analytes of interest.

The term “biological sample” as used herein relates to a sample derived from a biological fluid, cell, tissue, or organ of a human subject, comprising a mixture of biomolecules including proteins, peptides, lipids, nucleic acids, and the like. Generally, although not necessarily, the sample is a blood sample such as a whole blood sample, a serum sample, or a plasma sample.

As used herein, the term “cell-free nucleic acid” encompasses both cell-free DNA and cell-free RNA, where the cell-free DNA and cell-free RNA may be in a cell-free fraction of a biological sample comprising a body fluid. The body fluid may be blood, including whole blood, serum, or plasma. In most instances, the biological sample is a blood sample, and a cell-free nucleic acid sample, e.g., a cell-free DNA sample, is extracted therefrom using now-conventional means known to those of ordinary skill in the art and/or described in the pertinent texts and literature; kits for carrying out cell-free nucleic acid extraction are commercially available (e.g., the AllPrep® DNA/RNA Mini Kit and QIAmp DNA Blood Mini Kit, both available from Qiagen, or the MagMAX Cell-Free Total Nucleic Acid Kit and the MagMAX DNA Isolation Kit, available from ThermoFisher Scientific). Also see, e.g., Hui et al. Fong et al. (2009) Clin. Chem. 55(3):587-598.

The term “amplifying” as used herein refers to generating one or more copies, or “amplicons,” of a template nucleic acid, such as may be carried out using any suitable nucleic acid amplification technique, such as technology, such as PCR, NASBA, TMA, and SDA.

The terms “enrich” and “enrichment” refer to a partial purification of template molecules that have a certain feature (e.g., nucleic acids that contain 5-hydroxymethylcytosine) from analytes that do not have the feature (e.g., nucleic acids that do not contain hydroxymethylcytosine). Enrichment typically increases the concentration of the analytes that have the feature by at least 2-fold, at least 5-fold or at least 10-fold relative to the analytes that do not have the feature. After enrichment, at least 10%, at least 20%, at least 50%, at least 80% or at least 90% of the analytes in a sample may have the feature used for enrichment. For example, at least 10%, at least 20%, at least 50%, at least 80% or at least 90% of the nucleic acid molecules in an enriched composition may contain a strand having one or more hydroxymethylcytosines that have been modified to contain a capture tag.

The term “sequencing,” as used herein, refers to a method by which the identity of at least 10 consecutive nucleotides (e.g., the identity of at least 20, at least 50, at least 100 or at least 200 or more consecutive nucleotides) of a polynucleotide is obtained.

The terms “next-generation sequencing” (NGS) or “high-throughput sequencing”, as used herein, refer to the so-called parallelized sequencing-by-synthesis or sequencing-by-ligation platforms currently employed by Illumina, Life Technologies, Roche, etc. Next-generation sequencing methods may also include nanopore sequencing methods such as that commercialized by Oxford Nanopore Technologies, electronic detection methods such as Ion Torrent technology commercialized by Life Technologies, and single-molecule fluorescence-based methods such as that commercialized by Pacific Biosciences.

The term “read” as used herein refers to the raw or processed output of sequencing systems, such as massively parallel sequencing. In some embodiments, the output of the methods described herein is reads. In some embodiments, these reads may need to be trimmed, filtered, and aligned, resulting in raw reads, trimmed reads, aligned reads. The term “read” as used herein includes “read pair” for sequencing that uses paired reads to generate DNA fragments sequenced from both ends.

More generally, the term “detection” is used interchangeably with the terms “determining,” “measuring,” “evaluating,” “assessing,” “assaying,” and “analyzing,” to refer to any form of measurement, and include determining if an element is present or not. These terms include both quantitative and/or qualitative determinations. Assessing may be relative or absolute. “Assessing the presence of” thus includes determining the amount of a moiety present, as well as determining whether it is present or absent. Assessing the level at a hydroxymethylation biomarker locus refers to a determination of the degree of hydroxymethylation at that locus.

“Accuracy” refers to the degree of conformity of a measured or calculated quantity (a test reported value) to its accurate (or true) value. Clinical accuracy relates to the proportion of true outcomes (true positives (TP) or true negatives (TN)) versus misclassified outcomes (false positives (FP) or false negatives (FN)), and may be stated as a sensitivity, specificity, positive predictive values (PPV) or negative predictive values (NPV), or as a likelihood, or odds ratio, among other measures.

“Performance” is a term that relates to the overall usefulness and quality of a diagnostic or prognostic test, including, among others, clinical and analytical accuracy, other analytical and process characteristics, such as use characteristics (e.g., stability, ease of use), health economic value, and relative costs of components of the test. Any of these factors may be the source of superior performance and thus usefulness of the test, and may be measured by appropriate “performance metrics,” such as AUC, time to result, shelf life, etc. as relevant.

“Clinical parameters” or “clinical features” encompass all non-sample biomarkers of subject health status or other characteristics, such as, without limitation, lesion size; lesion location; patient age; patient weight; patient gender; patient ethnicity; family history; genetic mutations; and PD-L1 tumor staining result, which is currently used in the clinic to determine whether anti-PD-1 therapy is in order.

A “formula,” “algorithm,” or “model” is any mathematical equation, algorithmic, analytical, or programmed process, or statistical technique that takes one or more continuous or categorical inputs and calculates an output value, sometimes referred to as a “probability score” or “index value.” Non-limiting examples of “formulas” include sums, ratios, and regression operators, such as coefficients or exponents, biomarker value transformations and normalizations (including, without limitation, those normalization schemes based on clinical parameters, such as gender, age, or ethnicity), rules and guidelines, statistical classification models, and neural networks trained on historical populations.

Of particular use in combining hydroxymethylation levels at various biomarker loci and clinical parameters, optionally in further combination with other factors (e.g., non-hydroxymethylation biomarkers), are linear and non-linear equations and statistical classification analyses to determine the relationship between hydroxymethylation levels at the biomarker loci detected in a patient sample and the patient's likelihood of responding to treatment a particular colorectal cancer therapy. In panel and combination construction, of particular interest are structural and syntactic statistical classification algorithms, and methods of risk index construction, utilizing pattern recognition and machine learning features, including established techniques such as cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, and Hidden Markov Models, among others. Many such algorithmic techniques have been further implemented to perform both feature (loci) selection and regularization, such as in ridge regression, lasso, and elastic net, among others. Other techniques may be used in survival and time to event hazard analysis, including Cox, Weibull, Kaplan-Meier, and Greenwood models well known to those of skill in the art. Many of these techniques are useful either combined with a hydroxymethylation biomarker selection technique, such as forward selection, backwards selection, or stepwise selection, complete enumeration of all potential biomarker sets, or panels, of a given size, genetic algorithms, or they may themselves include biomarker selection methodologies. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other studies, or cross-validated in the study they were originally trained in, using such techniques as Bootstrap, Leave-One-Out (LOO) and 10-Fold cross-validation (10-Fold CV). At various steps, false discovery rates may be estimated by value permutation according to techniques known in the art.

“Likelihood,” in the context of one embodiment of the present invention, is the probability that a patient will respond or not respond to treatment with a particular colorectal cancer therapy, i.e., the “likelihood” refers to the probability score associated with possible recurrence or non-recurrence within a follow-up time period after treatment. In another embodiment, “likelihood” is the probability that a patient is responding or is not responding to treatment with a colorectal cancer therapy that is underway or that has been completed.

A “hydroxymethylation level” refers to the extent of hydroxymethylation within a hydroxymethylation biomarker locus. The extent of hydroxymethylation is normally measured as hydroxymethylation density, e.g., the ratio of 5hmC-containing cfDNA fragments to total cfDNA fragments within a nucleic acid region. Other measures of hydroxymethylation density are also possible, e.g., the ratio of 5hmC residues to total nucleotides in a nucleic acid region.

A “hydroxymethylation profile” or “hydroxymethylation signature” refers to a data set that comprises the hydroxymethylation level at each of a plurality of hydroxymethylation biomarker loci. The hydroxymethylation profile may be a reference hydroxymethylation profile that comprises composite a hydroxymethylation profile for a population of individuals with at least one shared characteristic, as explained elsewhere herein. The hydroxymethylation profile may also be a patient hydroxymethylation signature, constructed from the measurement of hydroxymethylation levels at each of a plurality of hydroxymethylation biomarker sites.

The “hydroxymethylation biomarkers” herein comprise loci selected for their relevance to the likelihood that a cancer patient will or will not experience cancer recurrence after surgical treatment of the tumor.

The term “locus” as used throughout this application refers to a site on a nucleic acid molecule, wherein the nucleic acid molecule may be single-stranded or double-stranded, and further wherein an individual locus (or multiple “loci”) may be of any length, thus including a single CpG site as well as a full-length gene, or across larger features such as topologically associated domains, including when several such loci are aggregated into groups such as related sequence motifs, other homologies or functional characteristics (regardless of their adjacency or topological relationship). The loci herein may be contained within a gene body; within an annotation feature outside of the gene body, such as a promoter, an enhancer, a transcription initiation site, a transcription stop site, or a DNA binding site, or a combination thereof; or within an untranslated region, or “UTR” (including 3′UTRs and 5′UTRs).

It should be noted that some of the individual hydroxymethylation biomarkers disclosed herein may not have significant individual significance in the evaluation of a patient's responsiveness to a particular colorectal cancer therapy, but when used in combination, and optionally in further combination with one or more other types of biomarkers and/or clinical parameters impacting on the evaluation and monitoring of a colorectal cancer tumor, become significant in discriminating as a method of the invention requires, e.g., between a subject who is likely to experience cancer recurrence within a selected follow-up time period and a subject who is not likely to experience recurrence within that follow-up time period.

For the purpose of this application, any two variables are considered to be “very highly correlated” when they have a Coefficient of Determination (R2) of 0.5 or greater. The present invention encompasses such functional and statistical equivalents to the presently disclosed hydroxymethylation biomarkers.

The term “correlate” as used herein in reference to two variables (e.g., two values, two sets of values, a value or value set and a disease state, a value or set of values and a risk associated with the disease state, or the like) indicates a tendency of the two variables to vary together. A “correlation” is a measure of the extent to which two or more variables fluctuate together. A positive correlation indicates the extent to which those variables increase or decrease in parallel. One example of a positive correlation is the relationship between a hydroxymethylation level at a hydroxymethylation biomarker locus, on the one hand, and the responsiveness of a colorectal cancer patient to treatment with a selected colorectal cancer therapy, on the other, when the hydroxymethylation level increases as the responsiveness of the subject increases. Conversely, a negative correlation would exist when the hydroxymethylation level at a hydroxymethylation biomarker locus decreases as a subject's responsiveness to treatment decreases.

The present invention relates, in part, to the discovery that certain biological markers, particularly epigenetic markers relating to DNA hydroxymethylation, correlate with the likelihood that a subject with colorectal cancer, lung cancer, or other cancer will respond either positively or negatively to treatment with a colorectal cancer therapy, a lung cancer therapy, or other cancer therapy, e.g., surgical treatment. In one embodiment, the method involves identifying 5hmC-containing fragments in a cfDNA sample obtained from the cancer patient and measuring the hydroxymethylation level of those fragments at each of a plurality of hydroxymethylation biomarker loci to generate a hydroxymethylation signature for a patient. This is followed by evaluating the extent of hydroxymethylation at each of the biomarker loci and then generating a probability score that the patient is likely to experience recurrence (or non-recurrence) of cancer during a follow-up time period after treatment with a selected cancer therapy.

The invention also enables a practitioner to determine the effectiveness of a colorectal or other cancer therapy being administered to a subject with colorectal or other cancer, including subjects with metastatic disease; to diagnose colorectal or other cancer in a patient who has not yet had a tumor identified; to assess the stage of an identified tumor; to predict whether a healthy individual is likely to develop colorectal or other cancer, to identify the risk that an identified colorectal or other tumor will develop into cancer; and to identify a change in the size, stage, grade, or degree of invasiveness of a cancerous colorectal or other tumor.

“Colorectal cancer,” also known as bowel cancer, colon cancer, or rectal cancer, is the presence of a cancerous lesion in the colon or rectum. As used herein, the term includes colorectal cancers regardless of cause, stage, or histopathologic characteristics. While most colorectal cancers are adenocarcinomas, colorectal tumors may also include lymphomas, adenosquamous cell carcinomas, squamous cell carcinomas. The “colorectal cancer patient,” as the term is used herein, refers to any living individual who has been diagnosed with colorectal cancer, via imaging, biopsy, or other known means, and refers to the intended subject of a colorectal cancer analysis and evaluation as discussed in detail herein.

The term “lung cancer” herein refers to any cancer of the lung, such as non-small-cell lung cancers including adenocarcinomas, squamous cell carcinomas, and large cell carcinomas; small-cell lung carcinomas; adenosquamous carcinomas; carcinoid tumors; bronchial gland carcinomas; and sarcomatoid carcinomas. Non-small cell lung cancer (NSCLC) is the most prevalent form of lung cancer, with adenocarcinomas most prevalent among non-smokers. The lung cancer patient who is evaluated using the present methods may be at an early or late stage of the disease, have a tumor that exhibits strong or weak PD-L1 staining, exhibit different spirometry results, and the like. The “lung cancer patient” as the term is used herein, refers to any living individual who has been diagnosed with lung cancer, via imaging, biopsy, or other known means, and refers to the intended subject of a lung cancer analysis and evaluation as provided herein.

While treatment of colorectal cancer, lung cancer, or other cancer often involves surgical resection of an identified lesion, treatment may also involve chemotherapy, radiation therapy, targeted therapy, immunotherapy, or other therapies that are known to those of ordinary skill in the art or yet to be discovered. Any two or more of these therapies may be used in combination, e.g., surgery followed by treatment with adjuvant chemotherapy, surgery followed by radiation therapy, and the like.

Chemotherapy drugs used to treat cancers such as colorectal cancer and lung cancer include, without limitation, 5-fluorouracil (5-FU), capecitabine, irinotecan, and oxaliplatin, with the dose and dosage regimen selected by the managing physician to optimize treatment outcome. Targeted therapy drugs include those that target blood vessel formation, e.g., VEGF inhibitors such as bevacizumab, ramucirumab, ziv-aflibercept, and fruquintinib; those that target cancer cells with EGFR changes, such as the EGFR inhibitors cetuximab and panitumumab; those that target cells with BRAF gene changes such as the BRAF inhibitor encorafenib; those that target HER2-positive cancers such as trastuzumab, pertuzumab, tucatinib, lapatinib, and fam-trastuzumab deruxtecan; those that target cells with NTRK gene changes such as larotrectinib and entrectinib; those that target cells with RET gene changes such as selpercatinib; those that target cells with KRAS gene changes such as adagrasib and sotorasib; as well as other target therapy drugs (e.g., regorafenib, a multikinase inhibitor). “Immunotherapy” refers to any method for treating disease by activating or suppressing the immune system. Examples of immunotherapies useful in treating colorectal cancer patients, lung cancer patients, and other cancer patients include, but are not limited to, cellular therapies such as dendritic cell therapy; antibody therapy; and cytokine therapy (for example, treatment with an interferon or an interleukin). Most commonly, immunotherapy treatment involves administration of a therapeutic antibody that binds to and blocks an immune checkpoint receptor protein such as CTLA-4, PD-1, or the like. PD-1 is a key immune checkpoint receptor, expressed by activated T cells and B cells, and mediates immunosuppression. Two cell surface glycoprotein ligands for PD-1 have been identified, Programmed Death Ligand-1 (PD-L1) and Programmed Death Ligand-2 (PD-L2), which are expressed on antigen-presenting cells as well as many human cancers and have been shown to down-regulate T cell activation and cytokine secretion upon binding to PD-1. Representative antibodies that target PD-1, a PD-1 ligand (e.g., PD-L1) or other immune checkpoint receptors or ligands thereof, and which are encompassed by the immunotherapies referenced herein include, without limitation, atezolizumab (Tecentriq®, Genentech); necitumumab (Portrazza®, Eli Lilly); nivolumab (Opdivo®, Bristol-Myers Squibb); cemiplimab (Libtayo, Regeneron Pharmaceuticals); avelumab (Bavencio, EMD Serono): durvalumab (Imfinzi®, AstraZeneca); dostarlimab (Jemperli®, GlaxoSmithKline); retifanlimab (Zynyz®, Incyte); and pembrolizumab (Keytruda®, Merck). It is to be understood that the invention is not limited in this respect, however, and that “therapy” as the term is used herein refers to any therapeutic treatment that is intended to kill tumor cells and is potentially useful in the treatment of colorectal cancer, lung cancer, or other cancer, while “immunotherapy” herein refers to any therapeutic treatment that activates or suppresses the immune system and is potentially useful in the treatment of colorectal cancer.

### 2. Hydroxymethylation Biomarkers

The invention is directed, in part, to a method for identifying differentially hydroxymethylated sites in cfDNA to serve as hydroxymethylation biomarkers in predicting a probability that a colorectal cancer patient, lung cancer patient, or other cancer patient, will or will not remain recurrence-free during a follow-up time period after treatment with a selected cancer therapy. The method involves the following steps, with colorectal cancer used for purposes of illustration:
(a) obtaining an initial cfDNA sample from each of a plurality of colorectal cancer patients prior to beginning treatment with the colorectal cancer therapy;
(b) determining a baseline count T0 in CPM at each of a plurality of candidate hydroxymethylation biomarker loci in the initial cfDNA samples;
(c) treating the patients with the colorectal cancer therapy;
(d) after treating the patients and at the end of the follow-up time period, confirming recurrence or nonrecurrence of cancer to identify a first population of cancer recurrent patients PR and a second population of cancer nonrecurrent patients PNR;
(d) obtaining a subsequent cfDNA sample from each of the patients;
(e) determining a subsequent count TS at each of the candidate hydroxymethylation biomarker loci in the subsequent cfDNA samples;
(f) adopting as hydroxymethylation biomarker loci those candidate hydroxymethylation biomarker loci exhibiting a threshold p-value of less than 0.05 and a difference z of at least 1.5, wherein

$$z = ( Ts - {T}_{0} ) / T 0.$$

Genes that used to differentiate between PD (Bucket D) from NED (Bucket A) patients are from the MSigDB Hallmark gene sets:
HALLMARK WNT BETA CATENIN SIGNALING: Genes up-regulated by activation of WNT signaling through accumulation of beta catenin CTNNB1 (n=42);
HALLMARK MYC TARGETS V1: A subgroup of genes regulated by MYC (n=200);p
HALLMARK OXIDATIVE PHOSPHORYLATION: Genes encoding proteins involved in oxidative phosphorylation (n=200); and
HALLMARK G2M CHECKPOINT: Genes involved in the G2/M checkpoint, as in progression through the cell division cycle (n=200).

The GSEA/Molecular Signatures Database (MSigDB) gene sets HALLMARK_WNT_BETA_CATENIN-SIGNALING, HALLMARK_MYC_TARGETSV1, HALLMARK_OXIDATIVE_PHOSPHORYLATION, and HALLMARK_G2M_CHECKPOINT are set forth in Liberzon et al. (2015), “The Molecular Signatures Database (MSigDB) hallmark gene set collection,” Cell Syst. 1(6): 417-425), incorporated by reference herein for the foregoing information.

### 3. Determination of a Cancer Patient's Baseline Hydroxymethylation Signature

The hydroxymethylation biomarkers identified in the preceding section are used in connection with a method for determining a probability that a cancer patient, e.g., a colorectal cancer patient or lung cancer patient, will or will not experience cancer recurrence during a follow-up time period after treatment with a selected cancer therapy. The method involves: (a) obtaining a cfDNA sample from the patient, enriching for hydroxymethylated DNA in the sample, amplifying the hydroxymethylated DNA, and sequencing the amplified hydroxymethylated DNA in a manner that identifies 5hmC-containing fragments or sites in the DNA; (b) determining a baseline hydroxymethylation signature for the patient, i.e., a hydroxymethylation signature that is determined prior to treatment with a colorectal cancer therapy or other cancer therapy, by identifying the extent of hydroxymethylation in the 5hmC-containing fragments or sites at each of the hydroxymethylation biomarker loci, or a subset thereof, identified in the preceding section; and (c) using the hydroxymethylation signature, calculating a probability score representing the probability that the cancer patient will or will not experience cancer recurrence during the follow-up time period after treatment with the cancer therapy.

At the outset, then, a cfDNA sample is obtained from a colorectal or other cancer patient undergoing evaluation. Extraction of cfDNA from a blood sample can be carried out using any suitable technique, for example using the commercially available kits referenced in Part 1 of this Detailed Description. The cfDNA is then enriched, so that the concentration of the cfDNA is substantially increased, a virtual necessity because of the very low levels of cfDNA normally obtained. A generally preferred enrichment technique is described by Chowdury et al. (2024) J. Mol. Diagnostics 26 (10): 888-896; also see International Patent Publication WO 2017/176630 to Quake et al., both of which are incorporated herein by reference in their entireties. Briefly, an affinity tag is appended to 5hmC residues in a sample of cfDNA, and the tagged DNA molecules are then selectively removed by bonding to a functionalized solid support. An illustrative example of the method, as described in Quake et al., involves initially modifying end-blunted, adaptor-ligated double-stranded DNA fragments in the cell-free sample to covalently attach biotin, as the affinity tag, to 5hmC residues. This may be carried out by selectively glucosylating 5hmC residues with uridine diphospho (UDP) glucose functionalized at the 6-position with an azide moiety, a step that is followed by a spontaneous 1,3-cycloaddition reaction with alkyne-functionalized biotin via a “click chemistry” reaction. The DNA fragments containing the biotinylated 5hmC residues are adapter-ligated dsDNA template molecules that can then be pulled down with a solid support functionalized with a biotin-binding protein (e.g., avidin or streptavidin) in the enrichment step.

The captured cfDNA is then amplified without having been releasing from the support, resulting in a plurality of amplicons. Any suitable amplification technique may be employed (e.g., PCR, NASBA, TMA, SDA) although PCR is preferred.

Next, the patient cfDNA is sequenced; it will be appreciated that in this instance sequencing will only produce reads of 5hmC-containing fragments in the cfDNA, the consequence of the enrichment step described above. In some embodiments, a portion of the patient cfDNA sample may be separated from the sample prior to enrichment; this separated portion is not enriched for 5hmC-containing fragments, but is subjected to sequencing using WGS, the results of which may be combined with the 5hmC information in making a determination of the probability score.

Then, the hydroxymethylation levels in the sequenced 5hmC-enriched cfDNA are measured at each of a plurality of differentially hydroxymethylated biomarker loci selected in the previous section.

In the embodiment wherein the colorectal or other cancer patient is not yet undergoing treatment with a particular cancer therapy, a determination can be made as to whether the therapy is likely to be effective in treating the patient. This is done by “mapping” the identified 5hmC-containing sites in the baseline hydroxymethylation signature to the identified hydroxymethylation biomarker loci.

That is, the hydroxymethylation level in the identified 5hmC-containing fragments in the patient's cfDNA sample is determined at each of the plurality of hydroxymethylation biomarker loci, where each locus serves as a hydroxymethylation biomarker that, as noted above, is differentially hydroxymethylated with respect to the likelihood that a colorectal or other cancer patient will or will not respond to treatment with a particular colorectal or other cancer therapy. Information regarding hydroxymethylation levels is thus deduced from the sequence reads obtained. That is, the sequence reads are analyzed to provide a quantitative determination of which sequences are hydroxymethylated in the cfDNA and the level of hydroxymethylation. This may be done by, e.g., counting sequence reads or, alternatively, counting the number of original starting molecules, prior to amplification, based on their fragmentation breakpoint and/or whether they contain the same molecular UFI. The use of molecular UFI sequences (or “molecular barcodes” as they are sometimes called) in conjunction with other features of the fragments (e.g., the end sequences of the fragments, which define the breakpoints) to distinguish between the fragments is known. See Casbon (2011) Nucl. Acids Res. 22 e81 and Fu et al. (2011) Proc. Natl. Acad. Sci. USA 108:9026-31), among others. Molecular barcodes are also described in U.S. Patent Publication Nos. 2015/0044687, 2015/0024950, and 2014/0227705, and in U.S. Pat. Nos. 8,835,358 and 7,537,897, as well as a variety of other publications.

The data set comprised of the patient cfDNA hydroxymethylation levels at each of a plurality of hydroxymethylation biomarker loci serves as the baseline hydroxymethylation signature for the patient.

A molecular UFI sequence is preferably incorporated into the adapters that are end-ligated to the cfDNA following extraction thereof. The adapters may be constructed so as to comprise an additional UFI sequence, e.g., a sample UFI sequence, a strand-identifier UFI sequence, or both.

Other methods of ascertaining the hydroxymethylation signature of DNA in the cell-free nucleic sample are described in International Patent Publication WO 2019/160994 A1 to Arensdorf et al. for “Methods for the Epigenetic Analysis of DNA, particularly Cell-Free DNA”; in co-pending U.S. patent application Ser. Nos. 16/275,237 and 17/118,234 to Arensdorf et al.; and in Liu et al. (2019) Nature Biotech. 37:424-29, all of which are incorporated by reference herein. These references are also useful in conjunction with an embodiment of the invention in which a patient's cfDNA methylation profile is identified in addition to the patient's cfDNA hydroxymethylation profile.

Both targeted and non-sequencing detection approaches after enrichment may also be used to quantitate specific hydroxymethylation biomarkers and loci of interest, if genome-wide coverage through shotgun sequencing is not required or desirable (generally for cost reasons). For example, after 5hmC enrichment, targeted PCR amplicons covering only specific regions may be generated from the 5hmC-enriched templates and employed as a narrower genome coverage approach, and used as input to sequencing or detected directly.

When a smaller number of discrete loci are of interest, the combination of these post-enrichment approaches with target amplification may also be an efficient way to reduce the number of sequencing reads (and sequencing costs) required for each sample, enabling further sample multiplexing per sequencing run and further reducing the sequencing costs required for each sample). In non-sequencing approaches, quantitative PCR or even hybridization assays could themselves be used as the quantitative readouts of the hydroxymethylation biomarkers (e.g., using direct fluorescence nucleotide labeling and microarray or other substrate capture and binding); such approaches are well known in the art, and frequently scaled to hundreds or even thousands of short amplicons.

In the present process, a 5hmC UFI sequence is added to the termini of the pulled down adapter-ligated dsDNA template molecules, so that the after amplification, pooling, and sequencing, information regarding hydroxymethylation profile can be deduced from the sequence reads obtained. That is, the sequence reads are analyzed to provide a quantitative determination of which sequences are hydroxymethylated in the cfDNA. This may be done by, e.g., counting sequence reads or, alternatively, counting the number of original starting molecules, prior to amplification, based on their fragmentation breakpoint and/or whether they contain the same molecular UFI. The use of molecular UFI sequences (or “molecular barcodes” as they are sometimes called) in conjunction with other features of the fragments (e.g., the end sequences of the fragments, which define the breakpoints) to distinguish between the fragments is known. See Casbon et al. (2011), among others. Molecular barcodes are also described in U.S. Patent Publication Nos. 2015/0044687, 2015/0024950, and 2014/0227705, and in U.S. Pat. Nos. 8,835,358 and 7,537,897, as well as a variety of other publications.

Other methods of ascertaining the hydroxymethylation profile of DNA in the cell-free nucleic sample are described in International Patent Publication WO 2019/160994 A1 to Arensdorf et al. for “Methods for the Epigenetic Analysis of DNA, particularly Cell-Free DNA” and in U.S. Patent Publication No. 2017/0298422 to Song et al., both incorporated by reference herein. These references are also useful in conjunction with an embodiment of the invention in which the present 5-hydroxymethylation determination and analysis further includes the detection of a cfDNA methylation profile in addition to the cfDNA hydroxymethylation profile.

The Arensdorf et al. methodology described in WO 2019/160994 can be implemented as follows:

Dual-Biotin Technique: After a cell-free nucleic acid sample has been extracted from a biological sample, with cfDNA having been adapter-ligated, 5hmC residues in the cfDNA are selectively labeled with an affinity tag, e.g., a biotin moiety as explained earlier herein. Biotinylation can be carried out by selective functionalization of 5hmC residues via βGT-catalyzed glucosylation with uridine diphosphoglucose-6-azide followed by a click chemistry reaction to covalently attach an alkyne-functionalized biotin moiety as explained previously. An avidin or streptavidin surface (e.g., in the form of streptavidin beads) is then used to pull out all of the dsDNA template molecules biotinylated at the 5hmC locations, which are then placed in a separate container for UFI sequence attachment during amplification. The remaining dsDNA template molecules in the supernatant are fragments that either have 5mC residues or have no modifications (the latter group including cDNA generated from cfRNA). A TET protein is then used to oxidize 5mC residues in the supernatant to 5hmC; in this case, a TET mutant protein is employed to ensure that oxidation of 5mC does not proceed beyond hydroxylation. Suitable TET mutant proteins for this purpose are described in Liu et al. (2017) Nature Chem. Bio. 13:181-191, incorporated by reference herein. The βGT-catalyzed glucosylation followed by biotin functionalization is then repeated. The fragments so marked—biotinylated at each of the original 5mC locations—are pulled down with streptavidin beads. The bead-bound DNA fragments are then barcoded—with a UFI sequence than used in the first step, i.e., a 5mC UFI sequence—during amplification. Unmodified DNA fragments, i.e., fragments containing no modified cytosine residues, now remain in the supernatant. If desired, sequence-specific probes can be used to hybridize to unmethylated DNA strands. The hybridized complexes that result can be pulled out and tagged with a further UFI sequence during amplification, as before.

Pyridine Borane Methodology: This is an alternative to the dual biotin technique, and is also a bisulfite-free process. The method relies on the use of pyridine borane, or an alternative, equally effective organic borane, to convert 5-carboxylcytosine (5caC) and 5-formylcytosine (5fC)—both of which can be generated from 5mC and 5hmC—to dihydrouracil (DHU). As DHU residues are read as thymine (T), while 5mC and 5hmC are read as C, the difference between parallel sequence reads enables the determination of DHU locations, which in turn indicates the location of 5mC and 5hmC locations.

In one embodiment, the pyridine borane method enables the identification of 5hmC locations in adapter-ligated target DNA in a cell-free sample. Initially, target DNA is oxidized with an oxidizing reagent that converts 5hmC to 5caC or 5fC, where the oxidizing reagent selected does not affect 5mC. Oxidation may be carried out enzymatically, although chemical oxidizing reagents are preferred in this embodiment. Examples of suitable chemical oxidizing agents for use in carrying out the aforementioned conversion include, without limitation: a perruthenate anion in the form of an inorganic or organic perruthenate salt, including metal perruthenates such as potassium perruthenate (KRuO₄), tetraalkylammonium perruthenates such as tetrapropylammonium perruthenate (TPAP) and tetrabutylammonium perruthenate (TBAP), and polymer supported perruthenate (PSP); and inorganic peroxo compounds and compositions such as peroxotungstate or a copper (II) perchlorate/TEMPO (2,2,6,6-tetramethyl-1-piperidinyloxy) combination. The modified DNA containing 5caC or 5fC in lieu of 5hmC is then treated with an organic borane effective to reduce, deaminate, and either decarboxylate or deformylate the oxidized 5hmC and provide DHU in place thereof. The DHU-containing DNA is amplified and sequenced to provide 5hmC-indicative sequence reads, insofar as the sequence reads can be readily compared to standard sequence reads obtained for the target DNA, where the change from C in the standard sequence reads to a T in the 5hmC-indicative sequence reads indicates a 5hmC location.

In another embodiment, the pyridine borane methodology is used to identify 5mC locations in adapter-ligated target DNA in a cell-free sample. In this case, 5hmC residues in the target DNA are, at the outset, tagged with an affinity tag that enables removal of 5hmC-containing fragments from the sample. For instance, 5hmC residues may be selectively glucosylated with uridine diphospho (UDP) glucose functionalized at the 6-position with an azide moiety, a step that is followed by a spontaneous 1,3-cycloaddition reaction with alkyne-functionalized biotin via a “click chemistry” reaction. The resulting biotinylated DNA target molecules can then be separated from the sample with a solid support functionalized with a biotin-binding protein (e.g., avidin or streptavidin). Remaining DNA in the sample will contain 5mC, but not 5hmC. In the next step, 5mC residues in the remaining DNA are enzymatically oxidized to 5caC or 5fC, followed by treatment with pyridine borane to convert the oxidized 5mC residues to DHU. A preferred enzyme useful as the oxidizing agent is a Ten-Eleven Translocation Enzyme (TET) family enzyme or a “TET catalytically active fragment” as defined in U.S. Pat. No. 9,115,386, the disclosure of which is incorporated by reference herein. A preferred TET enzyme in this context is TET2; see Ito et al. (2011) Science 333(6047):1300-1303. Following amplification and sequencing, a comparison of the sequence reads obtained with the standard sequence reads for the target DNA indicates the location of 5mC residues in the sample DNA, as the change from C to T (resulting from DHU substitution for 5mC) indicates a 5mC location.

In a further embodiment, the pyridine borane technique can be implemented to detect the locations of both 5mC and 5hmC residues in a single cell-free DNA sample. The method involves, for a first fraction of a cell-free DNA sample comprising adapter-ligated target DNA,
(a) blocking 5hmC residues with a blocking reagent to yield blocked 5hmC residues;
(b) enzymatically oxidizing 5mC residues to provide oxidized 5mC residues selected from 5caC, 5fC, and combinations thereof;
(c) converting the oxidized 5mC residues to DHU by treatment with pyridine borane, thereby providing first fraction DNA comprising blocked 5hmC residues and DHU at 5mC locations; and
(d) amplifying and sequencing the first fraction DNA to provide first fraction sequence reads in which the blocked 5hmC residues read as C and DHU reads as T.

Glucosylation is effective as a blocking technique, in which case the blocking reagent may be β-glucosyltransferase and the resulting blocking group on the 5hmC residues is glucose.

For a second fraction of the same sample, the method further involves:
(e) oxidizing 5hmC residues with an oxidizing reagent effective to convert 5hmC residues to oxidized 5hmC residues without modifying 5mC residues, wherein the oxidized 5hmC residues are selected from 5caC, 5fC, and combinations thereof; and
(f) converting the oxidized 5hmC residues to DHU by treatment with pyridine borane, thereby providing second fraction DNA comprising unmodified 5mC residues and DHU at 5hmC locations;
(g) amplifying and sequencing the second fraction DNA to provide second fraction sequence reads in which the unmodified 5mC residues read as C and DHU reads as T; and
(h) comparing the first fraction sequence reads with the second fraction sequence reads to identify 5mC and 5hmC locations in the template DNA.

See, e.g., Liu et al. (2019).

The organic borane may be characterized as a complex of borane and a nitrogen-containing compound selected from nitrogen heterocycles and tertiary amines. The nitrogen heterocycle may be monocyclic, bicyclic, or polycyclic, but is typically monocyclic, in the form of a 5- or 6-membered ring that contains a nitrogen heteroatom and optionally one or more additional heteroatoms selected from N, O, and S. The nitrogen heterocycle may be aromatic or alicyclic. Preferred nitrogen heterocycles herein include 2-pyrroline, 2H-pyrrole, 1H-pyrrole, pyrazolidine, imidazolidine, 2-pyrazoline, 2-imidazoline, pyrazole, imidazole, 1,2,4-triazole, 1,2,4-triazole, pyridazine, pyrimidine, pyrazine, 1,2,4-triazine, and 1,3,5-triazine, any of which may be unsubstituted or substituted with one or more non-hydrogen substituents. Typical non-hydrogen substituents are alkyl groups, particularly lower alkyl groups, such as methyl, ethyl, n-propyl, isopropyl, n-butyl, isobutyl, t-butyl, and the like. Exemplary compounds include pyridine borane, 2-methylpyridine borane (also referred to as 2-picoline borane), and 5-ethyl-2-pyridine. Further information concerning these organic boranes and reaction thereof to convert oxidized 5mC residues to DHU may be found in the Arensdorf patent publication cited above.

Biotin/Native 5mC Enrichment Method: This is an alternative to the dual biotin technique, and begins with biotinylation of 5hmC residues in adapter-ligated DNA fragments, followed by avidin or streptavidin pull-down. Here, however, instead of modifying the methylated DNA that remains in the supernatant, an anti-5mC antibody or an MBD protein is used to capture and pull down native 5mC-containing fragments. This technique is less preferred herein, insofar as it does not result in the generation of dsDNA template molecules that can be amplified, pooled, and sequenced with other dsDNA template molecules deriving from the same sample.

### 4. Probability Score Calculation

In order to generate a probability score, the measured hydroxymethylation levels in the patient's cfDNA baseline hydroxymethylation signature are input into a computer-generated predictive model that comprises a trained machine learning model. The predictive model is used to generate the probability score, i.e., a score representing the likelihood that the patient will or will not experience cancer recurrence during a follow-up time period after treatment with a selected colorectal cancer therapy. As before, the 5hmC molecular response score approach may also be used, in addition to or as an alternative to the foregoing method.

More specifically, in order to calculate the probability score or 5hmC-based molecular response score, the methods of the invention include statistical analyses and mathematical modeling used to analyze high-dimensional and multimodal biomedical data, i.e., the data obtained using the present methods for comparing hydroxymethylation profiles. The methods make use of one or more objective algorithms, models, and analytical methods that include mathematical analyses based on topographic, pattern-recognition based protocols, e.g., support vector machines (SVM), linear discriminant analysis (LDA), naive Bayes (NB), and K-nearest neighbor (KNN) protocols, as well as other supervised learning algorithms and models, such as Decision Tree, Perceptron, and regularized discriminant analysis (RDA), and similar models and algorithms well-known in the art (Gallant, 1990).

Statistical analyses include determining mean (M), e.g., geometric mean, standard deviations (SD), Geometric Fold Change (FC), and the like. Whether differences in hydroxymethylation levels are deemed significant may be determined by well-known statistical approaches, typically by designating a threshold for a particular statistical parameter, such as a threshold p-value (e.g., p<0.05), a threshold S-value (e.g., ±0.4, with S≤−0.4 or S>0.4), or other value, at which differences are deemed significant, for example when the level of biomarker hydroxymethylation in a hydroxymethylation profile is considered significantly increased or decreased, respectively, relative to the hydroxymethylation level at the same hydroxymethylation biomarker locus in a reference hydroxymethylation profile.

In one aspect, the methods of the invention apply the mathematical formulations, algorithms, or models to distinguish between normal and cancerous samples, and between various sub-types, stages, and other aspects of disease or disease outcome. In another aspect, the methods are used for prediction, classification, prognosis, and treatment monitoring and design.

For the comparison of hydroxymethylation levels or other values, data are compressed. Compression typically is by Principal Component Analysis (PCA) or a similar technique for visualizing the structure of high-dimensional data. PCA is used to reduce dimensionality of the data (e.g., measured expression values) into uncorrelated principal components (PCs) that explain or represent a majority of the variance in the data, such as about 50, 60, 70, 75, 80, 85, 90, 95 or 99% of the variance. PCA allows the visualization of biomarker levels and the comparison of hydroxymethylation profiles, such as between normal or reference samples and test samples. PCA mapping, e.g., 3-component PCA mapping is used to map data to a three-dimensional space for visualization, such as by assigning first, second, and third PCs to the x-, y-, and z-axes, respectively.

In some embodiments, there is a linear correlation between hydroxymethylation levels of two or more biomarkers. Pearson's Correlation (PC) coefficients may be used to assess linear relationships (correlations) between pairs of values, such as between hydroxymethylation levels of a biomarker. This analysis may be used to linearly separate distribution in expression patterns, by calculating PC coefficients for individual pairs of the biomarkers (plotted on x- and y-axes of individual Similarity Matrices). Thresholds may be set for varying degrees of linear correlation, such as a threshold for highly linear correlation of (R.sup.2>0.50, or 0.40). Linear classifiers can be applied to the datasets. In one example, the correlation coefficient is 1.0.

In some embodiments, Feature Selection (FS) is applied to remove the most redundant features from a dataset, such as a hydroxymethylation biomarker dataset. FS enhances the generalization capability, accelerates the learning process, and improves model interpretability. In one aspect, FS is employed using a “greedy forward” selection approach, selecting the most relevant subset of features for the robust learning models. (Peng et al., 2005). In some embodiments, SVM algorithms are used for classification of data by increasing the margin between the n data sets (Cristianini and Shawe-Taylor, 2000).

Analytic classification of the hydroxymethylation biomarkers herein can be made according to predictive modeling methods that set a threshold for determining the probability that a sample (e.g., a cfDNA sample obtained from a patient) belongs to a given class (e.g., increased likelihood that a colorectal cancer patient will respond to treatment with a particular colorectal cancer therapy). The probability preferably is at least 50%, or at least 60%, or at least 70%, or at least 80% or higher. Classifications also can be made by determining whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

The predictive ability of a model can be evaluated according to its ability to provide a quality metric, e.g., AUROC (area under the ROC curve) or accuracy, of a particular value, or range of values. Area under the curve measures are useful for comparing the accuracy of a classifier across the complete data range. Classifiers with a greater AUC have a greater capacity to classify unknowns correctly between two groups of interest. In some embodiments, a desired quality threshold is a predictive model that will classify a sample with an accuracy of at least about 0.7, at least about 0.75, at least about 0.8, at least about 0.85, at least about 0.9, at least about 0.95, or higher. As an alternative measure, a desired quality threshold can refer to a predictive model that will classify a sample with an AUC of at least about 0.7, at least about 0.75, at least about 0.8, at least about 0.85, at least about 0.9, or higher.

As is known in the art, the relative sensitivity and specificity of a predictive model can be adjusted to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity can be at least about 0.7, at least about 0.75, at least about 0.8, at least about 0.85, at least about 0.9, at least about 0.95, at least about 0.98, at least about 0.99, or higher.

Raw data can be initially analyzed by measuring the hydroxymethylation level for each biomarker. The data can be manipulated, for example, raw data can be transformed using standard curves, and the average of multiple measurements, if made, can be used to calculate the average and standard deviation for each patient. The data are then input into a selected predictive model, which will classify the sample. The resulting information can be communicated to a patient or health care provider, usually in the form of a written report.

In one embodiment, hierarchical clustering is performed in the derivation of a predictive model, where the Pearson correlation is employed as the clustering metric. One approach is to consider a dataset as a “learning sample” in a problem of “supervised learning.” CART is a standard in applications to medicine (Singer, Recursive Partitioning in the Health Sciences (Springer, 1999)) and can be modified by transforming any qualitative features to quantitative features, sorting them by attained significance levels, and a selected regularization method then applied (e.g., elastic net or lasso).

In some embodiments, the predictive models include Decision Tree, which maps observations about an item to a conclusion about its target value (Zhang et al., “Recursive Partitioning in the Health Sciences,” in Statistics for Biology and Health (Springer, 1999). The leaves of the tree represent classifications and branches represent conjunctions of features that devolve into the individual classifications.

The predictive models and algorithms may further include Perceptron, a linear classifier that forms a feed forward neural network and maps an input variable to a binary classifier (Gallant (1990), supra). In this model, the learning rate is a constant that regulates the speed of learning. A lower learning rate improves the classification model, while increasing the time to process the variable (Markey et al. (2002).

As explained earlier herein, the invention provides a method for determining the likelihood that an individual with colorectal cancer will or will not experience cancer recurrence within a follow-up time period after treatment with a particular colorectal cancer therapy. The invention additionally provides methods for determining the likelihood that a colorectal cancer patient undergoing treatment or having finished a treatment protocol is benefiting from the treatment. The invention thus encompasses diagnostic, prognostic, and predictive uses of hydroxymethylation profiles, as well as uses in patient monitoring, evaluation of treatment options, and evaluation of treatment efficacy, wherein, in each method of use, the hydroxymethylation profile generated is optionally combined with additional biomarker information and/or clinical parameters. All of the methods involve the generation of a hydroxymethylation profile comprising measurements of hydroxymethylation levels at each of a plurality of hydroxymethylation biomarker loci, where the loci are selected so as to exhibit differential hydroxymethylation in colorectal cancer patients who are responders or nonresponders to treatment with a particular therapy.

Among the provided predictive and diagnostic/monitoring methods are those which employ statistical analysis and biomathematical algorithms and predictive models to analyze the detected hydroxymethylation information. Some embodiments include methods and systems for analyzing the hydroxymethylation information in classification, staging, prognosis, treatment design, evaluation of treatment options, prediction of outcomes (e.g., predicting development of metastases), and the like.

### 5. Patient Monitoring During Therapy

In a related embodiment, a method is provided for monitoring a colorectal or other cancer patient during colorectal or other cancer therapy. This involves, at the outset, obtaining hydroxymethylation monitoring data for the cancer patient by: (i) obtaining a cfDNA sample from the patient, enriching for hydroxymethylated DNA in the sample, amplifying the hydroxymethylated DNA, and sequencing the amplified hydroxymethylated DNA in a manner that identifies 5-hydroxymethylcytosine (5hmC)-containing fragments or sites in the DNA; and (ii) measuring hydroxymethylation levels in the sequenced cfDNA at each of a plurality of hydroxymethylation biomarker loci, wherein each hydroxymethylation biomarker locus exhibits an increase or decrease in hydroxymethylation in a manner that correlates with a likelihood that the patient is or is not responding to the cancer therapy.

Also provided are methods that use evaluation of hydroxymethylation levels at the biomarker loci in treatment response prediction and patient monitoring, including evaluation of a patient's response to treatment and patient-specific or individualized treatment strategies. In some embodiments, the methods are used in conjunction with treatment, for example, by generating a hydroxymethylation profile weekly or monthly before, after, or at the time of treatment. As the hydroxymethylation levels at certain biomarker loci correlate with the progression of disease, ineffectiveness or effectiveness of treatment, and/or the recurrence or lack thereof of disease, the regular generation of hydroxymethylation profiles within an extended monitoring or treatment period is useful. In some aspects, the information obtained may indicate that a different treatment strategy is preferable. Thus, provided herein are therapeutic methods, in which biomarker evaluation is performed prior to treatment, and then used to monitor therapeutic effects.

More specifically, at various points in time after initiating or resuming treatment, significant changes in hydroxymethylation levels at one or more of the biomarker loci may be seen, indicating that a therapeutic strategy, e.g., surgery, chemotherapy, or the like, is or is not successful, or that a change in therapeutic approach is advised. In some embodiments, the therapeutic strategy is changed following a hydroxymethylation analysis, such as by adding a different therapeutic intervention, either in addition to or in place of a prior approach, by increasing or decreasing the aggressiveness or frequency of the approach, or by stopping or reinstituting a treatment regimen.

### 6. Analysis of Multiple Feature Types

The method of the invention may also involve a consideration of one or more additional feature types in combination with the 5-hydroxymethylation analyses described above. That is, the probability score representing the likelihood that a patient will respond to colorectal or other cancer therapy or is responding to colorectal or other cancer therapy takes into account not only the patient's 5-hydroxymethylation levels at specific 5hmC biomarker loci but also one or more additional feature types that correlate with the likelihood that the patient will respond to treatment with a colorectal cancer therapy. The additional feature may be an additional type of biological marker. That is, the cell-free DNA sample obtained from the patient, in addition to being analyzed in terms of hydroxymethylation levels at various loci, may also be analyzed with respect to biomarkers such as methylation levels; DNA fragment size and fragment size distribution; cell-free DNA concentration in a patient sample, corresponding to cfDNA plasma concentration ([p-cfDNA]); RNA analysis such as T cell-inflamed gene expression profile (GEP) (see Cristescu et al. (2018)); changes in circulating tumor DNA (ctDNA) count; the surrogate markers for tumor neoantigens microsatellite instability-high (MSI-H), deficient mismatch repair (dMMR), and tumor mutational burden, which can be measured from tissue samples or plasma samples (see Thompson et al. (2021) and Bindal et al. (2021)); expression of the immune suppression biomarkers LAG3 and IDO-1; regulatory T-cell count (Tregs); myeloid derived suppressor cell count; inflammation gene signatures; tumor infiltrating effector cells; lymphocyte count; microbiome composition; germline mutations; and the like.

Patient-specific clinical parameters may also be considered in combination with the hydroxymethylation analysis. These covariates include factors such as lesion size; lesion grade; lesion stage; lesion location; patient age; patient weight; patient body mass index (BMI), patient gender; patient ethnicity; cigarette smoking history; and exposure or lack of exposure to a known carcinogen.

In one embodiment, an ensemble model, e.g., a stacked ensemble model, is used to combine multiple datasets and machine learning techniques to predict a colorectal cancer patient's likelihood of responding to treatment with a particular colorectal cancer therapy or to determine whether a colorectal cancer patient who is undergoing treatment is responding to the colorectal cancer therapy used. The model uses 5hmC count data within various annotated regions across the genome such as a gene body, promoter, 5′ UTR, 3′ UTR, enhancer, intron, exon, LINE, SINE, or the like. Each annotated region is considered a feature set and incorporated into the stacked ensemble. In addition to the 5hmC features, additional feature values are used in this embodiment that are determined from one or more additional feature types. For example, feature values can be determined from additional feature types such as cfDNA fragment size and size distribution, copy number variation, and cell-free DNA plasma concentration from a WGS library constructed for the cell-free DNA sample obtained from a patient.

In a representative embodiment, a WGS library derived from the patient cfDNA sample is GC-corrected and processed to determine within approximately 1 MB, 2 MB, 4 MB, 5 MB, or even 8 MB windows the number of fragments in two or more different size ranges, e.g., two, three, four, five, or more different size ranges within the fragment size distribution obtained. Examples include two size ranges of 100-150 bp and 150-220 bp; two size ranges of 100-150 bp and 150-300 bp; two size ranges of 100-150 bp and 150-400 bp; two size ranges of 120-155 bp and 155-200 bp; 50-150 bp and 150-400 bp; three size ranges of 100-160 bp, 160-200 bp, and 200-220 bp; three size ranges of 50-152 bp, 153-240 bp, and 241-1000 bp, and the like.

In another representative embodiment, instead of or in addition to a WGS library derived from the patient cfDNA sample, the number of fragments in two or more different fragment size ranges is taken from only those fragments having 5hmC sites, which can be isolated from the patient cfDNA sample as explained in Part 2 of this Section. That is, although the foregoing description pertains to fragment size evaluation in a WGS library, the 5hmC-containing fragments can be evaluated in the same way, and used in addition to or instead of the WGS fragment size analysis.

Although the ratio of the number of large fragments to the number of small fragments can be employed as a single feature, it is preferred that the absolute number of fragments in a particular size range be used as an individual feature, such that, for example, for two size ranges, the number of fragments in the first size range (e.g., 100-150 bp) serves as a first feature and the number of fragments in the second size range (e.g., 150-220 bp) serves as a second feature. As another example, for three size ranges, the number of fragments in the first size range (e.g., 100-160 bp), the number of fragments in the second size range (e.g., 160-200 bp), and the number of fragments in the third size range (e.g., 200-220 bp) serve as three distinct features.

The foregoing features, i.e., the number of fragments within each of two or more specific size ranges, may be combined with at least one other feature type, in addition to the patient hydroxymethylation profile, in the analysis that follows. Copy number variation (CNV) is one such additional feature type. This may be readily determined from the GC-corrected WGS library. For instance, the number of reads of length 50-1000 bp, or another selected length, can be mapped in individual windows, e.g., 100 kb windows, along the genome to support detection of CNV. Cell-free DNA concentration in the patient sample can serve as yet an additional feature to be combined with hydroxymethylation profile and at least one of CNV and number of fragments in different size bins. Concentration of cfDNA in the patient sample can be readily determined by methods described in the pertinent literature or known to those of ordinary skill in the art. See, e.g., Chen et al. (2021) Nature Portfolio 11:5040, incorporated herein by reference.

After normalizing by total counts for each feature type, i.e., number of fragments in each of two or more size ranges elastic net regression models are built using glmnet. The elastic net mixing ratio α can be optimized, for instance, using k-fold (e.g., 5-fold, 10-fold, greater than 10-fold) cross validation (e.g., set to 0.01, 0.1, 0.5, or the like) for each feature set. The regularization parameter λ is optimized at run time per feature set, again using k-fold (e.g., 5-fold, 10-fold, or greater than 10-fold) cross validation.

The models built for all feature types—e.g., number of fragments in each of two or more size ranges, CNV, plasma cfDNA concentration, and 5hmC profiles—are combined together using a final elastic net fit with a predetermined elastic net mixing ratio (e.g., α=0.01, 0.1, 0.5, or the like) in a stacked ensemble fashion. The stacked ensemble combines the models by using the individual predictive scores from each separate model as a feature vector, then fitting for coefficients that weight the scores from each model. By way of illustration rather than limitation, the non-zero coefficients from the individual models can roughly comprise: 60-90% hydroxymethylation profile and 10-40% number of fragments within at least two size ranges. When CNV and cfDNA concentration are included, the relative weighting may be, as an example, 60-90% hydroxymethylation profile, 1-20% number of fragments within at least two size ranges, 1-20% CNV, and 1-20% cfDNA concentration. In one specific example, with cfDNA concentration omitted, the relative weighting may be 75-85% hydroxymethylation profile, 14-24% CNV, and 1% fragmentation.

A new sample can then be scored as follows. First, the 5hmC and WGS libraries are processed to prepare the feature vectors used by the individual elastic net models which are input into the full stacked ensemble model. The individual elastic net model predictive scores are computed from the appropriate (5hmC or WGS) feature vector. Then, those scores are passed into the full stacked ensemble model as input to generate a final probability score.

### Example 1

The invention is predicated, in part, on the discovery of a method for identifying baseline differential 5hmC biomarkers in plasma that can distinguish between cancer patients, e.g., CRC patients, who are likely to remain recurrence-free after treatment, on the one hand, and cancer patients, e.g., CRC patients, who are likely to exhibit recurrence after treatment, e.g., within two years of treatment. The experimental work documented herein is directed to a representative subcategory of the aforementioned method, wherein a method is provided for identifying baseline differential 5hmC biomarkers in plasma that can distinguish between mCRC patients who are likely to remain recurrence-free after curative-intent surgery and mCRC patients who are likely to exhibit recurrence, i.e., between “responders” and “nonresponders.”

### A. Methods

### (i) Clinical Cohorts and Study Design:

This work was performed using plasma obtained from subjects with late-stage mCRC who had undergone surgery to remove cancerous tissue and tumors. The subjects were in one of two groups: patients who had exhibited no recurrence at the two-year point (designated NED for “no evidence of disease”) or who had relapsed within two years surgery (designated PD for “progressive disease”).

To identify the potential for 5hmC-based biomarkers to provide information on post-surgical recurrence in mCRC patients, patient cfDNA was first isolated from plasma, then subjected to a 5hmC enrichment assay in which 5-hydroxymethylated cfDNA fragments were pulled down with a highly specific and sensitive chemical click reaction followed by DNA library preparation. Whole genome libraries were prepared from the same input cfDNA material. Genomic regions enriched for 5hmC were determined by peak detection using MACS2 (https://github.com/taoliu/MACS). Details of the procedures used are as follows:

### (ii) Plasma Collection:

Whole blood specimens obtained by routine venous phlebotomy in Becton-Dickinson Vacutainer EDTA tubes according to the manufacturer's protocol. The tubes were maintained at 15° C. to 25° C. until plasma isolation. Plasma was isolated within 24 hr of phlebotomy by centrifugation of whole blood at 1600×g for 10 min at room temperature, followed by transfer of the plasma layer to a new tube for centrifugation at 1600×g for 10 min. Plasma was then aliquoted and stored at −80° C.

(iii) Cell-Free DNA Isolation:

Cell-free DNA (cfDNA) was isolated from plasma using the method described in applicant's International Patent Application Publication No. WO 2023/235614 A1 for “Predicting and Determining Efficacy of a Lung Cancer Therapy in a Patient,” the disclosure of which is incorporated by reference herein. Specifically, cfDNA was isolated using the MagMAX® cell-free DNA isolation kit (Thermo Fisher Scientific, Waltham, MA) following the manufacturer's protocol with automated runs on HAMILTON STAR liquid handlers (HAMILTON Company, Reno, NV) using the MagMAX magnetic beads. During this procedure, plasma was incubated with Proteinase K and 20% SDS at 60° C. for 20 minutes followed by cooling. Next, cfDNA was bound to the magnetic beads and washed with a Thermo Fisher Scientific proprietary wash buffer and with 80% ethanol. Finally, cfDNA was eluted in 75 μl elution buffer. All cfDNA eluates were quantitated using Molecular Devices' Spectramax® Plate Readers using the PicoGreen® dsDNA quantitation assay (Thermo Fisher Scientific). TapeStation® 4200 capillary electrophoresis (Agilent Technologies, Santa Clara, CA) was employed to ensure the absence of contaminating high molecular weight DNA emanating from white blood cell lysis.

(iv) 5-Hydroxymethylcytosine (5hmC) Enrichment Assay and 5hmC/WGS Library Preparation:

5hmC-enriched libraries were prepared using the cell-free “5hmC-Seal” method described in International Patent Publication WO 2017/176630 to Quake et al., Song et al. (2011), and Han et al. (2016), the disclosures of which are incorporated by reference herein. Briefly, hMe-Seal is a low-input, whole-genome cell-free 5hmC sequencing method based on selective chemical labeling, in which β-glucosyltransferase is used to selectively label 5hmC with a biotin moiety via an azide-modified glucose for pull-down of 5hmC-containing DNA fragments for sequencing. In implementing hMe-Seal in the present case, the cfDNA was normalized to 10 ng total input for each assay and ligated to sequencing adapters, followed by selective labeling of 5hmC with β-GT, and affinity enrichment via selective pull-down of DNA fragments containing biotin-labeled 5hmC by binding to Dynabeads M270 Streptavidin (Thermo Fisher Scientific). PCR was then carried out directly on the beads to minimize sample loss during purification. All libraries were quantitated by Molecular Devices's SpectraMax Plate Readers using the PicoGreen® dsDNA quantitation assay (Thermo Fisher Scientific) and normalized to 1 ng/μl prior to pooling. Library pools were quantitated by Qubit dsDNA High Sensitivity Assay (Thermo Fisher Scientific) and normalized in preparation for sequencing.

### (v) DNA Sequencing and Alignment:

DNA sequencing was performed according to manufacturer's recommendations with 75 base-pair, paired-end sequencing using a NovaSeq instrument with version 2 reagent chemistry (Illumina, San Diego, CA). Data was collected using NovaSeq Control Software v1.8.1. Raw data processing and demultiplexing was performed using version 2.20.0.422 of the Illumina bcl2fastq software to generate sample-specific FASTQ output. Sequencing reads were aligned to the GRCh38reference genome using BWA-MEM 2 with default parameters and −K 200000000 (M. Vasimuddin, S. Misra, H. Li and S. Aluru, “Efficient Architecture-Aware Acceleration of BWA-MEM for Multicore Systems,” 2019 IEEE International Parallel and Distributed Processing Symposium (IPDPS), Rio de Janeiro, Brazil, 2019, pp. 314-324. Sequencing data quality was assessed using the Picard tool kit (Broad Institute).

### (vi) Peak Detection:

BWA-MEM read alignments were employed to identified regions or peaks of dense read accumulation that mark the location of a hydroxymethylated cytosine residue. Prior to identifying peaks, BAM files containing the locations of aligned reads were filtered for poorly mapped (MAPQ<30) and not properly paired reads using SAMtools (Li et al. (2009), “The Sequence Alignment/Map format and SAMtools,” Bioinform Oxf Engl 25:2078-9). 5hmC peak calling was carried out using MACS2 (https://github.com/taoliu/MACS) with a p-value cut off of 1.00e-5. Identified 5hmC peaks residing in “blacklist regions” as defined elsewhere (https://sites.google.com/site/anshulkundaje/projects/blacklists) and residing on chromosomes X, Y and mitochondrial genome were also removed using Bedtools (Quinlan et al. (2010), “BEDTools: a flexible suite of utilities for comparing genomic features,” Bioinformatics 26:841-842). Computation of genomic feature enrichment overlapping 5hmC peaks was performed using the software HOMER (http://homer.ucsd.edu/homer/) with default parameters.

(vii) Differential 5-Hydroxymethylation Analysis:

Raw counts over genes were normalized by transforming raw counts to log 2 (counts per million). Genes that map to sex chromosomes, and with weak representation genes (by requiring CPM>3 in at least 10 samples) were removed before analysis. Gene representation distributions were normalized using TMM (trimmed mean of M values). To identify differentially hydroxymethylated regions in non-responding patients relative to responding patients, the Wilcoxon sum test was applied to compare plasma hydroxymethylation profiles obtained at the baseline timepoint in non-responding versus responding patients. R package edgeR was used for differential analysis (see Robinson et al., 2010) followed by utilization of Benjamini's and Hochberg's procedure to account for multiple hypothesis testing and to quantify false discovery rate (FDR). Pre-ranked gene list based on fold change in 5hmC CPM over gene bodies was fed to the gene set enrichment analysis (GSEA) software and gene sets in mSigDB (Subramanian et al., 2005) with significant differential 5hmC representation in PD vs NED were identified at p-value<0.05.

The 5hmC-based molecular response (MR_{5hmc}) was also calculated, starting with an evaluation of

$${log}_{2} ( {T}_{R} / {T}_{0} )$$

at each 5hmC biomarker locus, wherein T₀ is the baseline CPM at each locus and T_{Q} is the CPM seen during therapy monitoring at time Q at each locus. The values of log 2 (T_{Q}/T₀) obtained at each locus are designated as either xᵢ (a positive value when there is a response or a negative value when there is non-response) or yᵢ (a positive value when there is non-response or a negative value when there is a response). That is, the xᵢ and yᵢ represent genomic loci exhibiting increased 5hmC that is either positively or negatively correlated with treatment response, respectively. Then, MR_{5hmc} is calculated as the difference of the means:

$${MR}_{5 hmC} = {μ}_{x} - {μ}_{y}$$

where μₓ and μ_{y} are the means of all xᵢ, i=1, . . . , nᵢ, and yⱼ, j=1, . . . , nⱼ, respectively (where in this example, nᵢ=129 and nⱼ=154). In this analysis, the selected loci had a p-value of less than 0.05 and a difference ξ of at least 1.5, wherein

$$ζ = ( {T}_{Q} - {T}_{0} ) / {T}_{0} .$$

It should be noted that while T_{Q} indicates the CPM at each 5hmC biomarker locus at some time point Q during treatment, the term T_{R} is used to refer to the specific time point at which imaging is done and therapy response evaluated according to the RECIST guidelines (i.e., responder=CR/PR or non-responder=PD).

(viii) Predictive Modeling:

To assess the feasibility of detecting patients' response to surgical treatment using the 5hmC profiling assay described above, cfDNA in plasma obtained from the mCRC patients was profiled. The intention was to use the samples to provide a set suitable for training a machine learning model that would detect a signal of progression. An ensemble of binomial models (“base learners”) was trained on the early versus late stage samples, where the feature vectors for the various binomial models were based on different genomic features derived from our assay. For example, the feature vectors used for one of the binomial models consisted of cpm counts of fragments mapped to gene bodies. Each base learner binomial model was trained using elastic net regularization, a means of performing feature reduction when the number of features exceeds the number of samples. See Friedman et al. (2010) J. Stat. Software 33(1): 1-22 for a description of the general elastic net procedure. Software implementation of these methods can be found at https://cran.r-project.org/web/packages/glmnet/index.html. Prior to fitting a base learner with elastic net, an initial filtering procedure was carried out which removed features with low variance. An elastic net logistic regression fit was performed using glmnet 40 with alpha, the mixing parameter, set to 0.01. The value of the elastic net regularization parameter lambda was set using cv.glmnet, which uses cross validation to pick an optimal value of lambda. After fitting the individual base learner binomials for each feature type vector, an elastic net binomial ensemble model was trained using the scores from the individual base learners setting alpha to 0.5 and again determining the value of lambda via cv.glmnet.

### B. Results:

Each patient's response to surgical treatment was determined by radiological imaging and categorized using the “Response Evaluation Criteria in Solid Tumors” (RECIST 1.1) guidelines (Eisenhauer et al., 2009). Patients were categorized as exhibiting recurrence (PD) or not exhibiting recurrence (NED) pursuant to the RECIST guidelines, as indicated in Table 1:

TABLE 1

| Non-relapsed (NED) ≥2 years | Relapsed (PD) <2 years |
|---|---|
| No evidence of disease (NED) at | Progressive Disease (PD) within 2 |
| 2 year-post-surgery (blood | years post-surgery (blood |
| collected at day of surgery) | collected at day of surgery) |
| with follow up data of at | |
| least 2 years | |

Baseline 5hmC signals were profiled for each sample and differential 5hmC signals were identified between non-relapsed (NED) and relapsed (PD) patients. The majority of the patients exhibited disease recurrence based on the clinical data. Table 2 indicates the number of quality control (QC)-passed patients in each group with QC results shown in Table 3:

TABLE 2

| | NED (n = 6) | PD (n = 48) |
|---|---|---|
| Female | 3 | 18 |
| Male | 3 | 30 |

TABLE 3
Quality Control Metrics

| Metric | Required | Threshold | QC_Results |
|---|---|---|---|
| [cfDNA], ng/μL | >= | 0.2 | PASS |
| [HMC lib], ng/μL | >= | 2 | PASS |
| [WGS lib], ng/μL | >= | 1 | PASS |
| HMC_unique_fragments | >= | 10,000,000.00 | PASS |
| HMC_percent_duplication | <= | 50 | PASS |
| HMC_number_genes_{—} | <= | 800 | PASS |
| zero_counts | | | |
| HMC_zero_CpG | <= | 0.064 | PASS |
| WGS_unique_fragments | >= | 10,000,000.00 | PASS |
| WGS_percent_duplication | <= | 50 | PASS |
| WGS_number_genes_{—} | <= | 800 | PASS |
| zero_counts | | | |
| WGS_mass_ratio_{—} | >= | 0.207 | PASS |
| error_factor | | | |

Following plasma collection, 5hmC enrichment, DNA sequencing and alignment, and the 5hmC enrichment assay described in Part A, sections (ii) through (v), peak detection was carried out using the methodology described in Part A, section (vi), with genomic regions enriched for 5hmC determined by peak detection using MACS2.

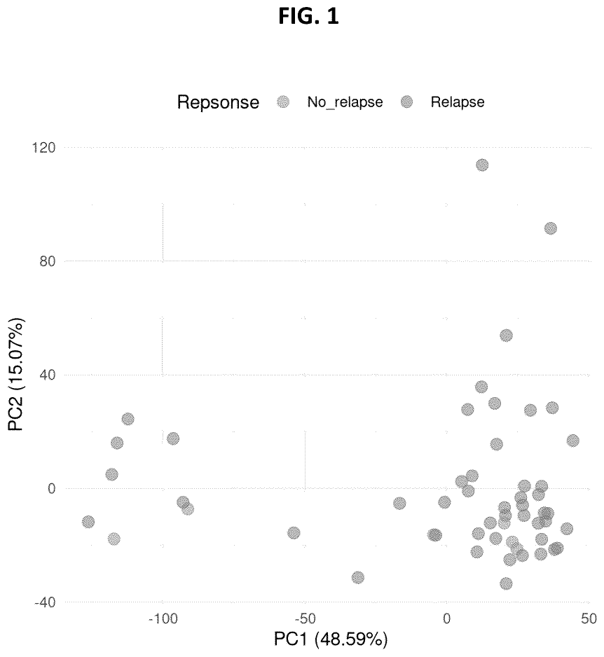

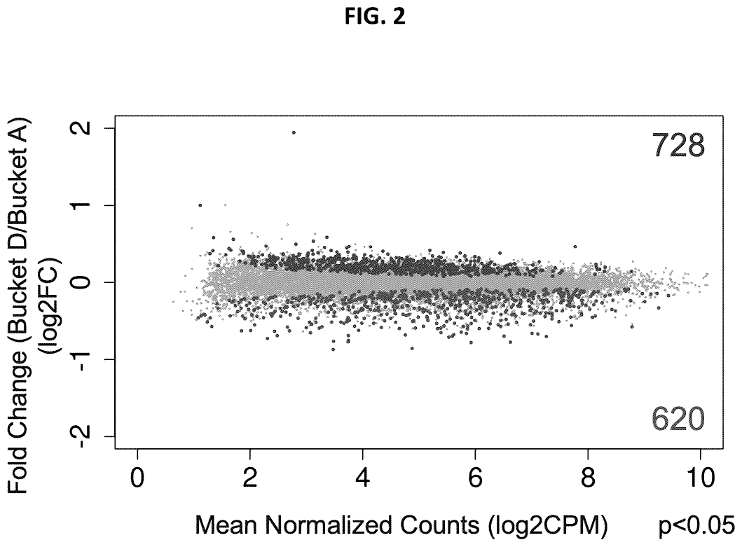

FIG. 1 is a tSNE plot using gene body 5hmC profiles (CPM) where Bucket A represents non-relapsed patients and Bucket D represents relapsed patients. To identify differential 5hmC marks (DhMRs), edgeR analysis was performed between the relapsed versus the non-relapsed patients. Due to the small sample size, none of the genes met the FDR<0.05 cutoff; see FIG. 2, showing the results of the edgeR analysis indicating genes with differential 5hmC gene body counts between relapsed (Bucket D) and non-relapsed (Bucket A) patients. However, 1,348 genes were identified with differential 5hmC levels (p<0.05) in relapsed patients compared to non-relapsed patients.

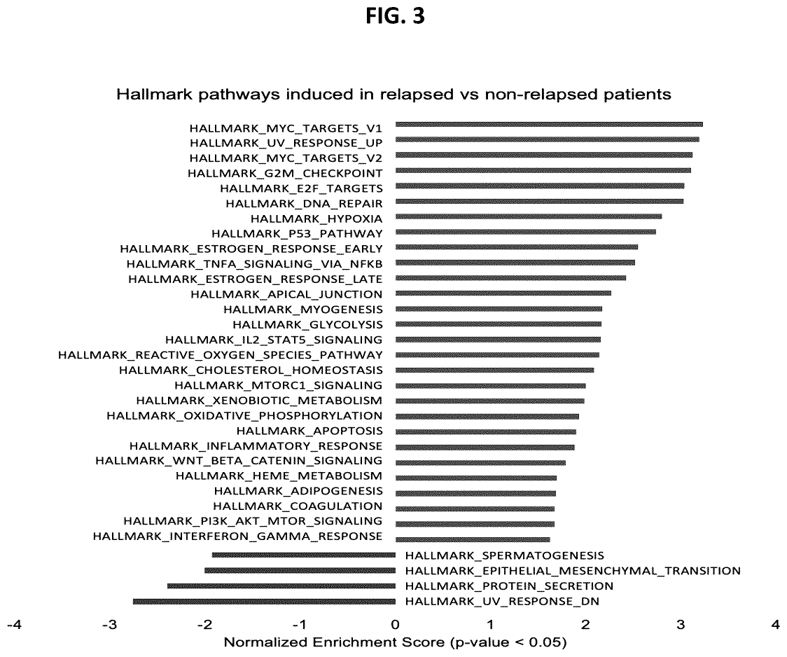

Next, gene set enrichment analysis (GSEA) was performed using the Hallmark and C6 databases to identify biological processes that can be distinguished by comparing the cfDNA 5hmC profiles of NED and PD patients. cfDNA 5hmC profiles in responding patients to pre-treatment cfDNA 5hmC profiles in non-responders. Tumor progression-relevant pathways, such as epithelial mesenchymal transition (EMT), G2M checkpoint, E2F targets, mTORC1 signaling, MYC targets, DNA repair, P53, and oxidative phosphorylation, were among the most differentially modulated gene sets, as seen in FIG. 3.

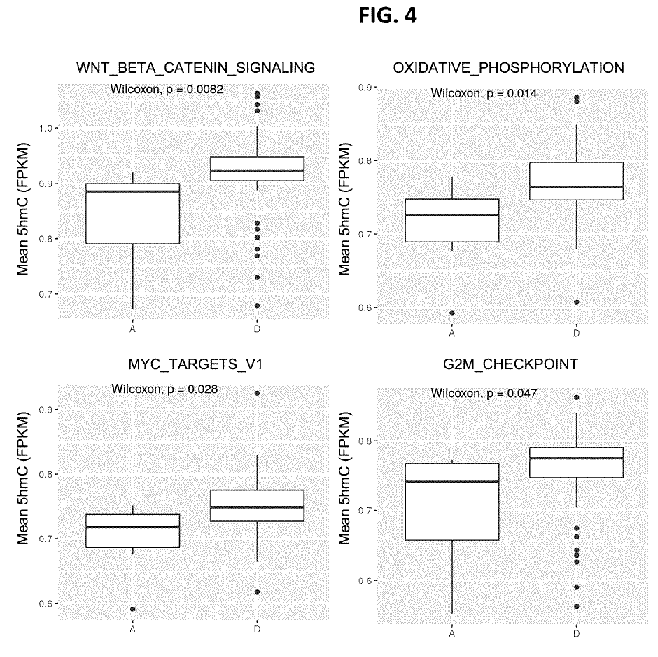

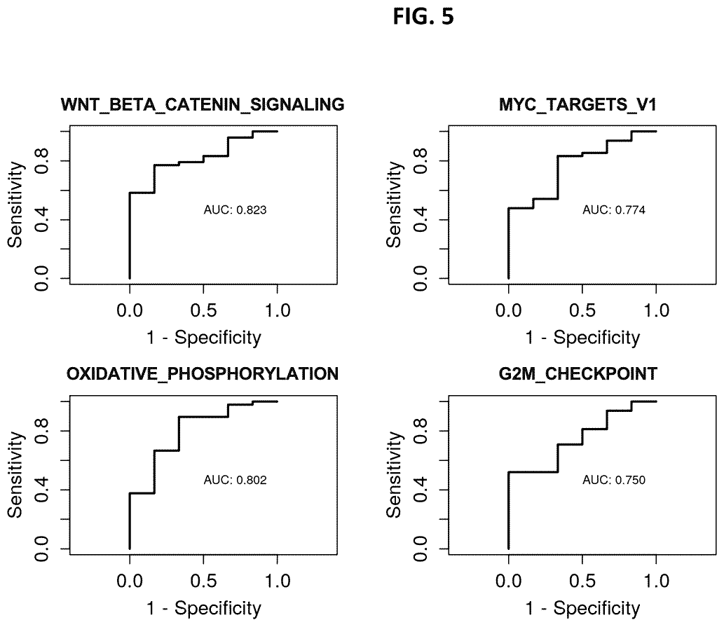

The Wnt/β-catenin signaling pathway has been linked to the initiation and progression of CRC (Bian et al., 2020) and overexpression of β-catenin in the nucleus has been associated with disease progression and a worse prognosis for CRC patients (Chen et al., 2013, Wong et al., 2004, Cheah et al., 2002). Inhibition of CRC progression has been achieved via degrading c-Myc (Wang et al., 2022), another pathway studied extensively in the context of CRC. However, correlation of the expression levels of proto-oncogene c-Myc (also referred to as MYC) with recurrence of disease or tumor progression has been controversial with some studies reporting positive correlation with improved survival (Smith and Goh, 1996; Toon et al., 2014, Lee et al., 2016), while others associating high c-Myc expression with significantly lower PFS and OS (Stropolli et al., 2020). Upregulated oxidative phosphorylation (OXPHO) levels were associated with tumorigenesis of CRC via promoting cell proliferation (Ren et al., 2023), and have been used to classify high-risk and low-risk CRC patients (Wang et al., 2014). Unexpectedly, 5hmC profiling over β-catenin, MYC, OXPHO and G2M checkpoint-involving gene sets showed higher abundance in relapsed patients relative to non-relapsed patients (FIG. 4). Area under the ROC (AUROC) curves suggested that these gene sets may predict disease recurrence and might further be combined to have a higher predictive performance (FIG. 5).

The differentially hydroxymethylated loci identified in cfDNA in distinguishing relapsed mCRC patients from mCRC patients who did not relapse not only distinguished between patient groups post-surgery but also have predictive value and can be used to guide the decision as to whether surgery should be performed on a particular patient.

### Example 2

### Detection of CRC: Development of Colorectal Cancer Model and Independent Validation of Colorectal Cancer Detection Test:

A colorectal cancer detection test was developed and validated using cfDNA 5hmC profiles in CRC and non-cancer patients. The training set was comprised of 294 colorectal cancer (59% late-stage) and 588 non-cancer plasma samples collected in Streck tubes. Samples were sex-, age-, BMI- and smoking status-matched to minimize background differences between cancer groups, as indicated in Table 4, below. WGS and 5hmC libraries were sequenced following cfDNA extraction. A logistic regression model based on 5hmC profiling of cfDNA and additional genomic features were utilized to develop a cancer detection test using plasma samples collected in Streck tubes. The machine learning algorithm capable of distinguishing patients with colorectal cancer from non-cancer subjects was validated on an independent cohort.

TABLE 4

| Sex | Female | Male |
|---|---|---|
| CRC | 124 | 170 |
| Non-cancer | 250 | 338 |

| Age & BMI | Average Age | STD Age | Average BMI | STD BMI |
|---|---|---|---|---|
| CRC | 61.4 | 11.2 | 30.3 | 9.79 |
| Non-cancer | 62.1 | 9.73 | 30.0 | 6.92 |

| Smoking Status | Current | Former | Never |
|---|---|---|---|
| CRC | 42 | 91 | 161 |
| Non-cancer | 86 | 184 | 318 |

| Cancer Stage | I | II | III | IV | 0 | NA |
|---|---|---|---|---|---|---|
| CRC | 49 | 62 | 99 | 75 | 5 | 4 |

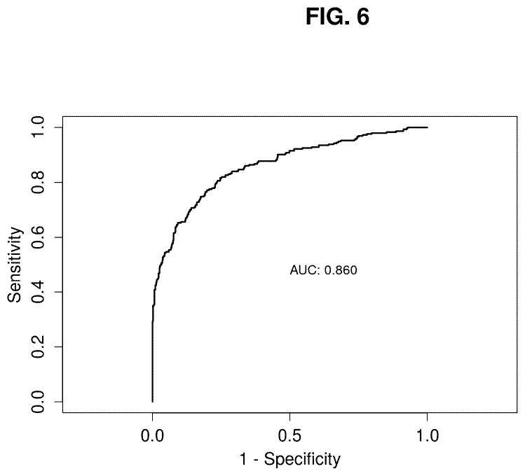

A binomial prediction algorithm was constructed using elastic net logistic regression combining predictors from both whole genome and 5hmC sequencing features. To simulate the performance of the algorithm on new data, the model was assessed using 10-fold cross validation, that is, with 10% of samples to be held from the training set and used for validation instead, training and testing are repeated 10-times. Performance was evaluated using held out 10% samples from each iteration. This resulted in an overall performance measured by area under the receiver-operating characteristic curve of 0.86 (FIG. 6). The overall test sensitivity for this 10-fold cross-validation analysis was 55.8% (95% confidence interval [CI]: 49.9%-61.5%) at 95% training specificity threshold, while test specificity was 93.7% (95% confidence interval [CI]: 91.4%-95.5%).

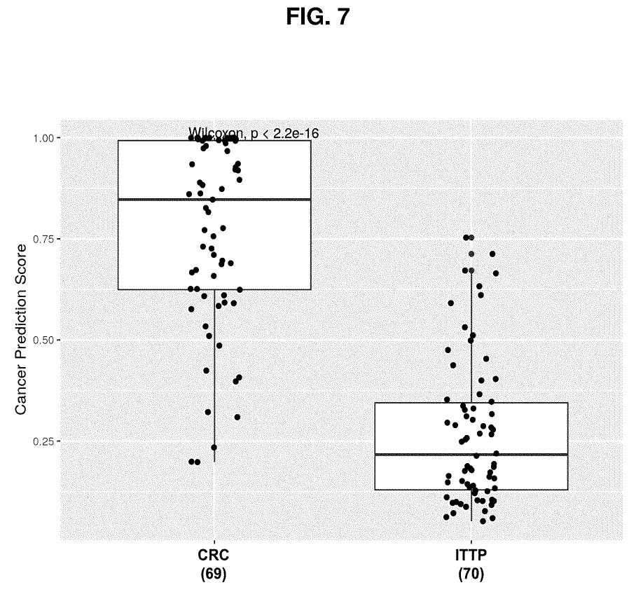

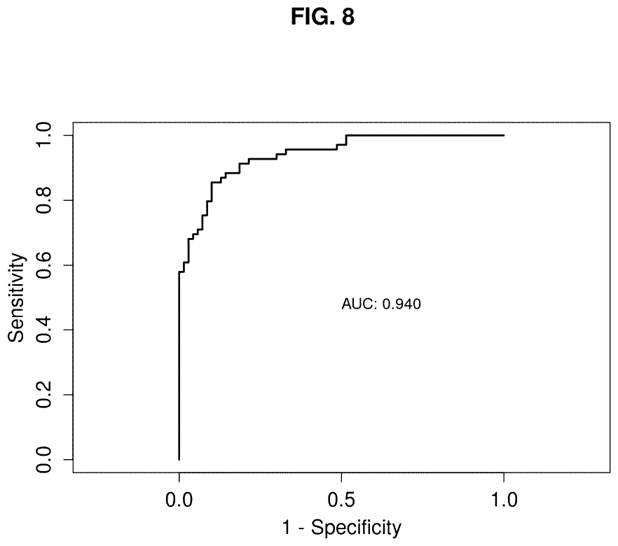

To further evaluate the performance of the CRC detection model, plasma samples collected in EDTA tubes and independently processed from the training set were scored with the CRC detection model. The independent validation data set was comprised of 69 CRC and 70 non-cancer plasma samples. CRC samples were distinguished from non-cancer samples (p-value=2.2e-16) when scored with the model generated using the Streck tubes (FIG. 7). Independent validation resulted in an area under the receiver-operating characteristic curve of 0.94, exceeding the performance obtained by 10-fold cross-validation (FIG. 8). Test sensitivity was 62.3% at 97.1% specificity using the training sensitivity threshold determined at 95% training specificity. Table 5 provides a list of sensitivity and specificity values from the samples at 80%, 85%, 90%, 92%, 95%, 97%, and 98% training specificity thresholds:

TABLE 5

| At % Training | | |
|---|---|---|
| Specificity | | |
| Threshold | Test Sensitivity, % | Test Specificity, % |
| 80 | 88.4 | 84.3 |
| 85 | 84.1 | 90 |
| 90 | 81.2 | 90 |
| 92 | 73.9 | 92.9 |
| 95 | 62.3 | 97.1 |
| 97 | 53.6 | 100 |
| 98 | 50.7 | 100 |

The examples that follow describe methodology and analysis that was used to show that tissue type signatures can be captured by plasma 5hmC profiling to indicate the presence of tumor-derived DNA and predict treatment response, without the need for tumor biopsy and RNA extraction, in cancers with solid tumors. To this end, genome-wide plasma 5hmC profiling over different tumor-indicative tissue-specific gene sets were analyzed in various cancer types. Cell-free DNA (cfDNA) was isolated from plasma collected in either Streck or EDTA tubes, and 5hmC-enriched and whole-genome sequences were generated using methods described previously; see, e.g., Xue et al. (2025), “5-Hydroxymethylcytosine analysis reveals stable epigenomic changes in tumor tissue that enable cancer detection in cell-free DNA,” Commun Biol 8: Article number 1613. To investigate the power of liquid biopsy in detecting cancer and monitoring treatment response, gene set enrichment analysis using C8 cell-type signature gene sets and genes identified to have predictive clinical value in tumor tissue were analyzed using cfDNA 5hmC profiling. Multiple cancer types were used to exemplify the use of plasma cfDNA 5hmC to be indicative of presence of cancer and treatment response through tissue-specific signatures. Additionally, plasma collected in either Streck or EDTA tubes were used to profile 5hmC levels over genes and colorectal cancer-specific models were generated for different tube types.

### Example 3

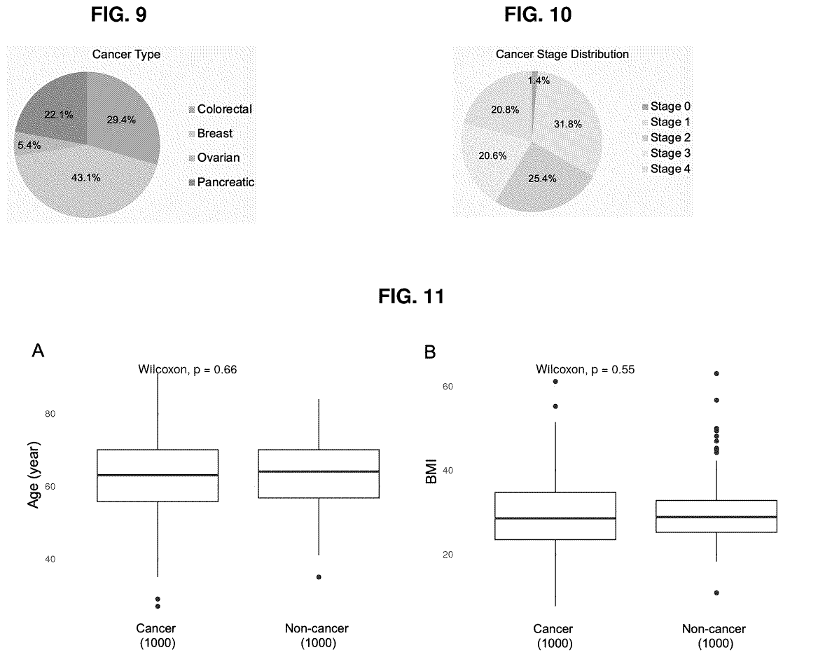

To assess the potential impact of cancer on plasma cfDNA 5hmC profiles over immune gene sets, blood collected in Streck tubes was obtained from 1,000 cancer samples with pathologic confirmation of disease and 1,000 non-cancer control samples. Blood samples from cancer subjects were collected prior to biopsy or surgical resection. The cancer cohort was composed of 431 breast (78% estrogen receptor (ER)+, 20.6% ER-, 6 subtype unknown), 294 colorectal, 54 ovarian and 221 pancreatic cancer samples (Table 6, FIG. 9).

TABLE 6

| Cohort | Size (N) | Mean Age (year) | Female (%) |
|---|---|---|---|
| Cancer | 1000 | 62.4 | 71.2 |
| Colorectal | 294 | 61.3 | 42.2 |
| Breast | 431 | 60.7 | 100.0 |
| Ovarian | 54 | 61.6 | 100.0 |
| Pancreatic | 221 | 66.1 | 46.6 |
| Non-cancer | 1000 | 62.9 | 71.2 |

| | Smoking Status (%) | | |
|---|---|---|---|
| | Current | Former | Never |
| Cancer | 13.9 | 28.8 | 57.3 |
| Non-cancer | 13.7 | 29.1 | 57.2 |

Cancer Stage Distribution (%)

| | Early-stage (0-II) | Late-stage (III-IV) |
|---|---|---|
| Cancer | 58.6 | 41.4 |
| Colorectal | 40.8 | 59.2 |
| Breast | 80.7 | 19.3 |
| Ovarian | 20.4 | 79.6 |
| Pancreatic | 48.4 | 51.6 |

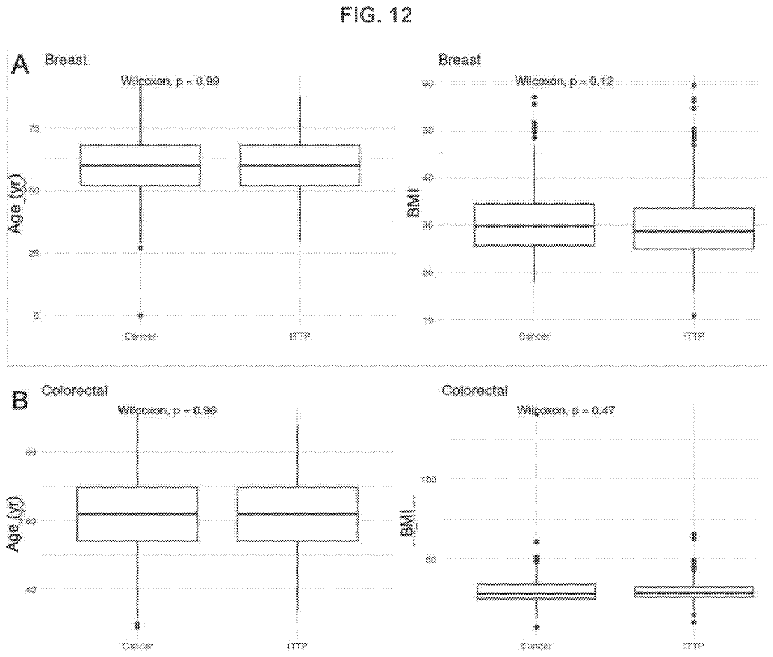

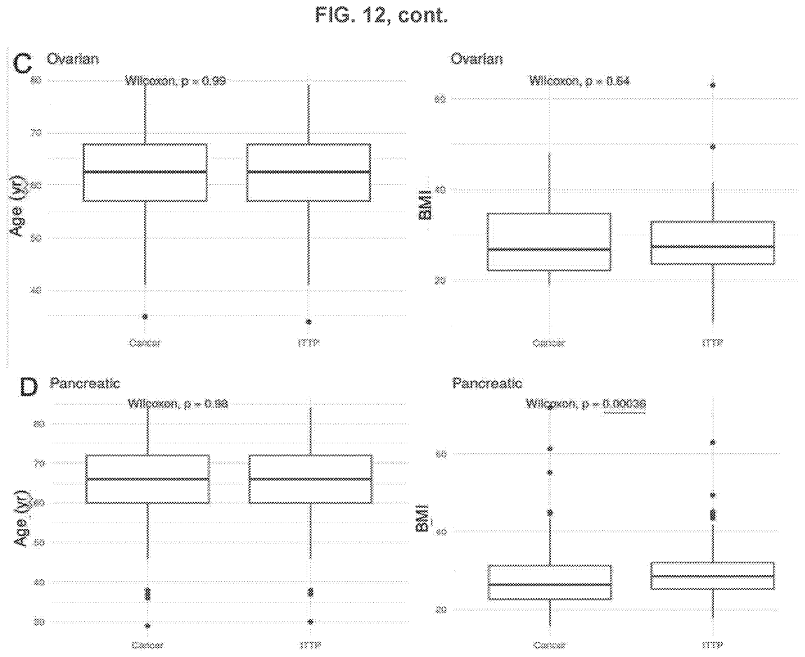

The majority of the cancer cohort was early stage, as shown in FIG. 10. Non-cancer samples for each cancer type evaluation were selected to match for sex, age, BMI, and smoking status for each cancer type (FIG. 11 and FIG. 12). FIG. 12 shows the age and BMI distributions of breast (A), colorectal (B), ovarian (C), and pancreatic (D) cancer cohorts along with their matched non-cancer cohorts (Wilcoxon test).

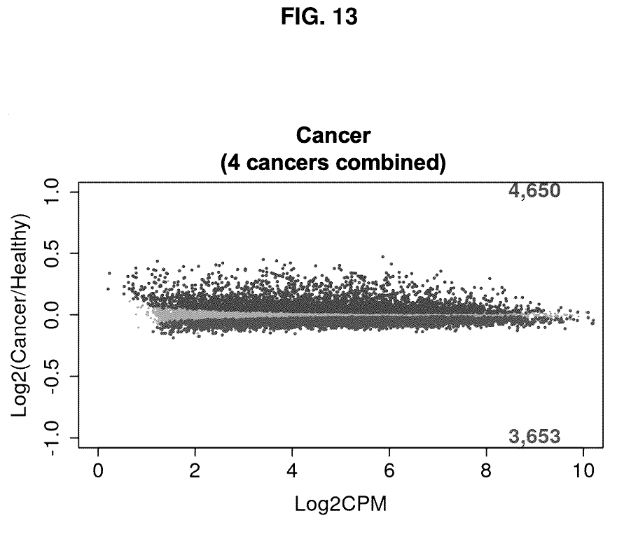

Initially, differential 5hmC analysis was performed over genes using all cancer samples compared to non-cancer controls. EdgeR analysis resulted in 8,303 DhMGs with 4,650 genes having more hydroxymethylation in cancer while 3,653 genes having less hydroxymethylation in cancer compared to non-cancer samples at FDR≤0.05. See FIG. 13, an MA plot of DhMG in cfDNA, comparing all cancer samples with non-cancer samples. The upper (red) and lower (blue) dots indicate increased or decreased 5hmC density in cancer compared to non-cancer samples (FDR≤0.05).

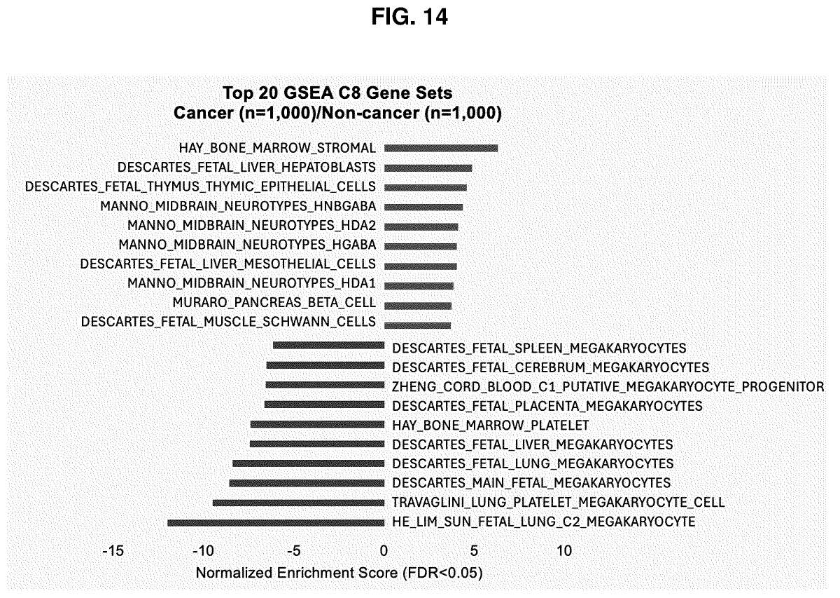

Next, gene set enrichment analysis was performed using C8 cell-type signature genes to gain insight into the biology of these DhMGs. Investigation of C8 gene sets enriched in the cfDNA 5hmC profiles in cancer samples relative to non-cancer controls revealed decreased hydroxymethylation in myeloid signatures. FIG. 14 shows the GSEA C8 normalized enrichment scores of the top C8-related positive and negative representative pathways in cancers relative to non-cancers. Gene sets with lowest 5hmC levels related to myeloid lineage in the cancer cohort belonged to megakaryocytes, as may be seen in FIG. 14.

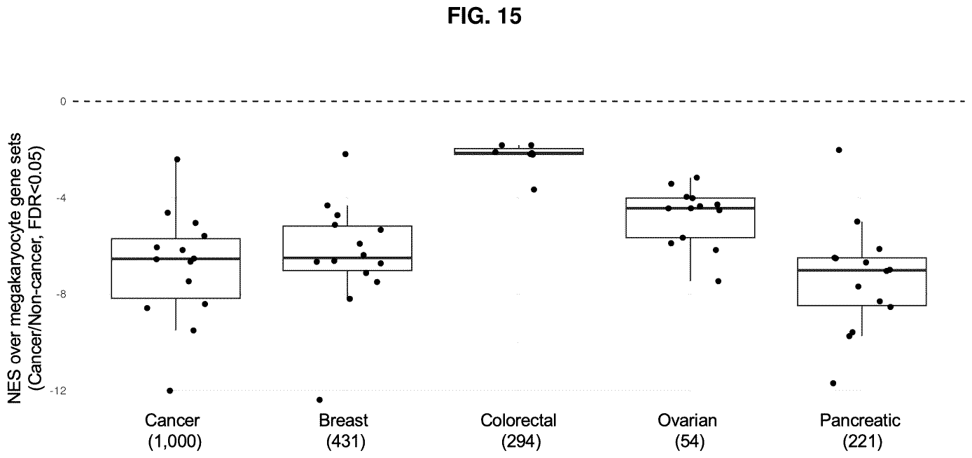

In contrast, genes with high 5hmC levels in cancer relative to non-cancer belong to those gene sets known to be involved in tumorigenesis, such as stroma and GABAergic signaling. These results show that plasma 5hmC profiling over cell type signatures can indicate the presence of cancer in blood. Because approximately 26% of cfDNA is contributed by megakaryocytes in healthy individuals (Moss et al., supra), reduced representation of 5hmC over megakaryocyte-derived cfDNA suggests higher proportions of tumor-derived DNA in the cfDNA pool, indicating the presence of cancer. Reduced 5hmC levels over all of the C8 gene sets associated with megakaryocytes (n=14) were observed in all cancers, and most of them were negatively enriched in individual cancer types relative to their matched non-cancer controls: breast, colorectal, ovarian and pancreatic; see FIG. 15, which provides the normalized enrichment scores (NES) of megakaryocyte gene sets between cancer and non-cancer samples. Each data point in a boxplot represents a megakaryocyte gene set (C8) with differential enrichment at FDR<0.05.

### Example 4

Indication of Cancer Using Cell-Type Signatures in CRC Patients with Solid Tumors:

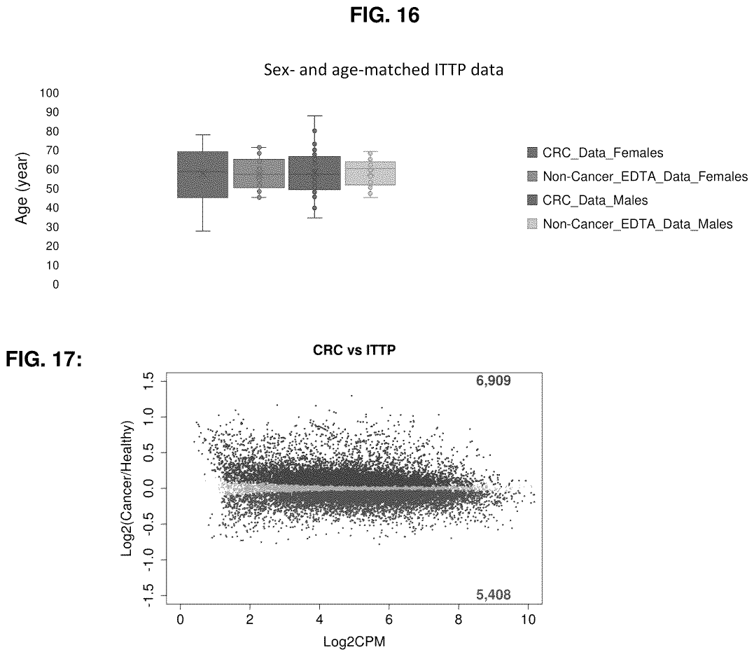

To assess the potential impact of cancer on plasma cfDNA 5hmC profiles over immune gene sets, blood collected in EDTA tubes was obtained from 70 late-stage colorectal cancer (CRC) patients with pathologic confirmation of disease and 70 non-cancer (ITTP) control samples. Blood samples from cancer subjects were collected prior to biopsy or surgical resection. Age and sex distributions were balanced between CRC samples and non-cancer samples (FIG. 16).

EdgeR 5hmC analysis over genes using CRC samples relative to non-cancer controls resulted in 12,317 differentially hydroxymethylated genes (DhMGs) with 6,909 genes having increased hydroxymethylation in CRC, while 5,408 genes having decreased hydroxymethylation in CRC compared to non-cancer samples at FDR<0.05. See the MA plot of DhMG in cfDNA provided in FIG. 17, comparing CRC samples versus non-cancer samples (FDR<0.05). Upper (red) and lower (blue) dots indicate increased or decreased 5hmC density in CRC compared to non-cancer samples, respectively.

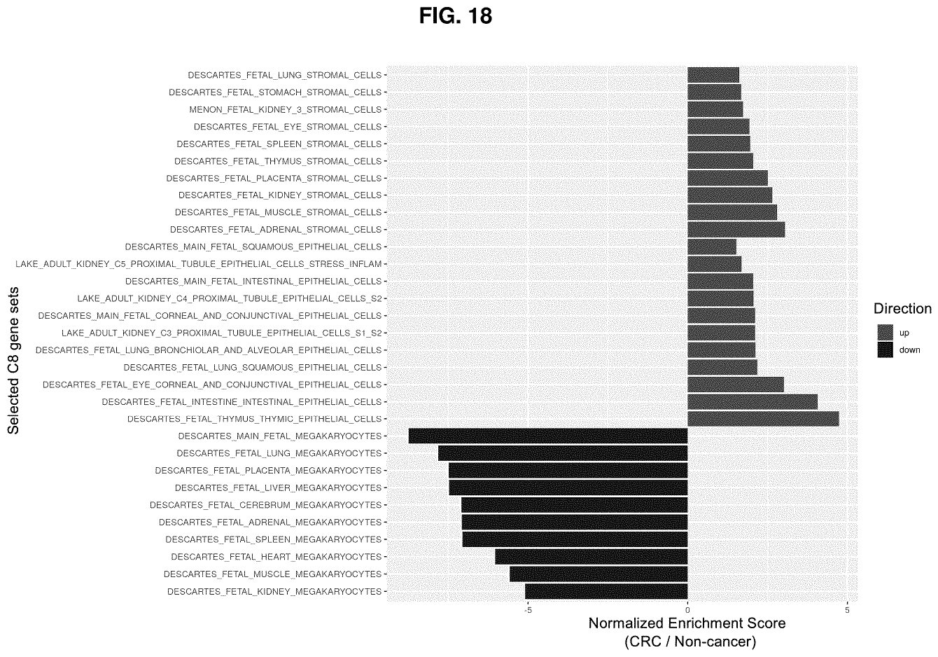

Next, gene set enrichment analysis using C8 cell-type signature genes was performed to gain biological insight. As in the preceding example, where different types of cancers were compared to non-cancers, 5hmC levels in genes related to megakaryocytes were found to be significantly lower in CRC patients compared to non-cancer patient samples, potentially suggesting a higher proportion of cfDNA derived from colon-tissue specific genes in cancer samples (FIG. 18). In contrast, genes implicated in epithelium and stroma, known to play important roles in promoting tumor progression, had higher 5hmC levels in CRC patients relative to non-cancer controls.

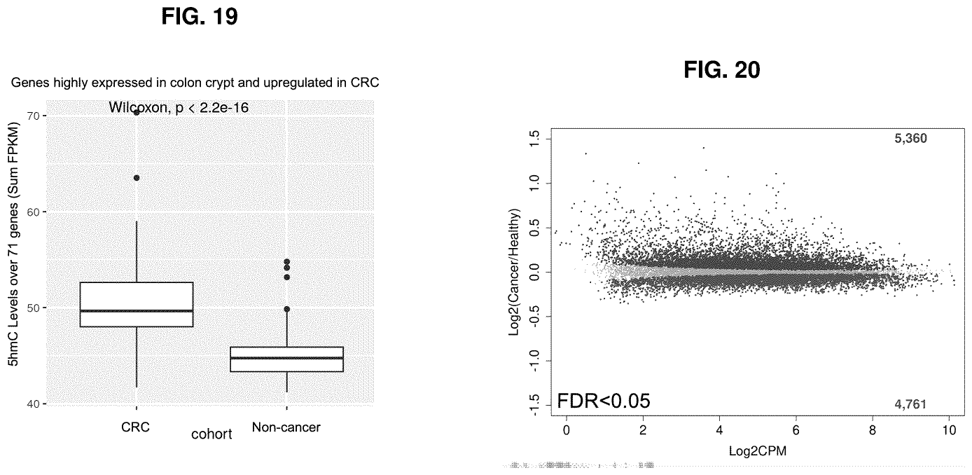

Because colon-specific gene sets are not included in C8 pathways, we sought to validate if CRC patients have increased plasma cfDNA 5hmC signals over the genes known to be colon specific. To this end, the overlap between the genes that are highly expressed in colon crypt identified by microarray analysis (Kosinski et al., 2007) and the genes that have elevated levels of 5hmC in CRC patients were examined. Approximately 25% (71/288) of colon crypt-specific genes showed higher 5hmC representation in CRC patients, suggesting colon-specific signals originated from the tumor tissue can be captured in plasma of CRC patients. See the boxplot of FIG. 19, which shows cumulative 5hmC levels (sum of FPKM) over 71 genes that are common between the colon crypt-specific gene set and genes with significantly increased 5hmC representation in CRC relative to non-cancer samples.

### Example 5

Evaluation of Plasma cfDNA Profiles to Reveal Differentially Modulated Pathways and Differentially Represented Cell Types in CRC Relative to Non-Cancer Samples:

An independent data set comprised of 66 CRC (Streck) and 147 non-cancer (Streck) samples were compared to reveal differentially hydroxymethylated genes. Gene body edgeR analysis resulted in 5,360 upregulated genes and 4,761 downregulated genes in CRC compared to age- and sex-matched non-cancer controls. FIG. 20 provides the MA plot from the edgeR analysis showing genes with differential 5hmC gene body counts in CRC patients compared to the non-cancer control.

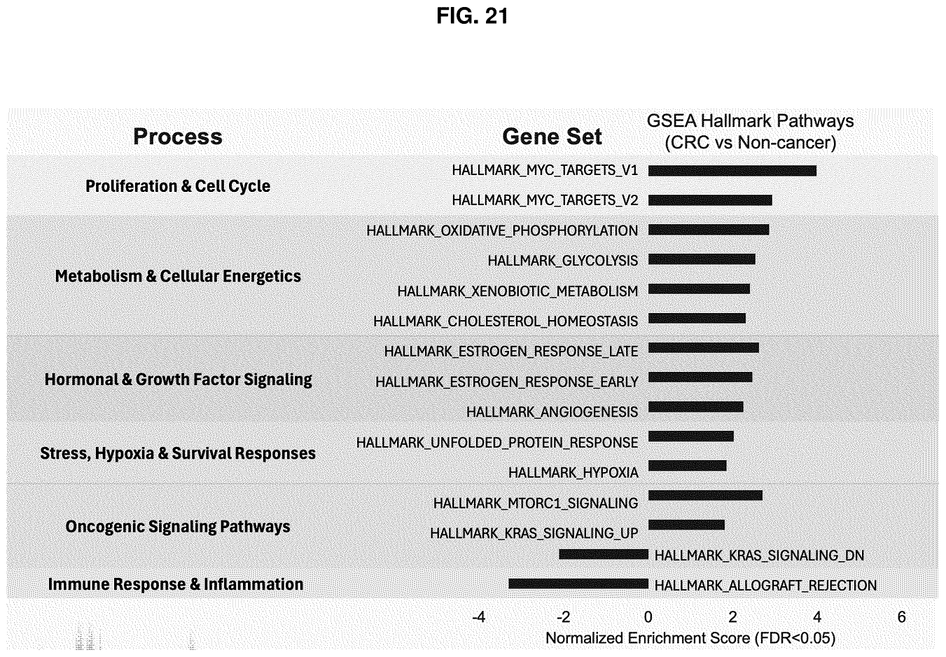

To gain deeper biological insight into altered pathways and contributing cell types, GSEA analyses were performed using MSigDB Hallmark, C6 (oncogenic signatures), and C8 (cell type signatures) gene sets. Hallmark pathway analysis revealed increased 5hmC gene body levels across pathways associated with enhanced cell proliferation, metabolic activity, stress responses, oncogenic signaling, and cell-cycle activation, consistent with progressive tumor biology; see FIG. 21.

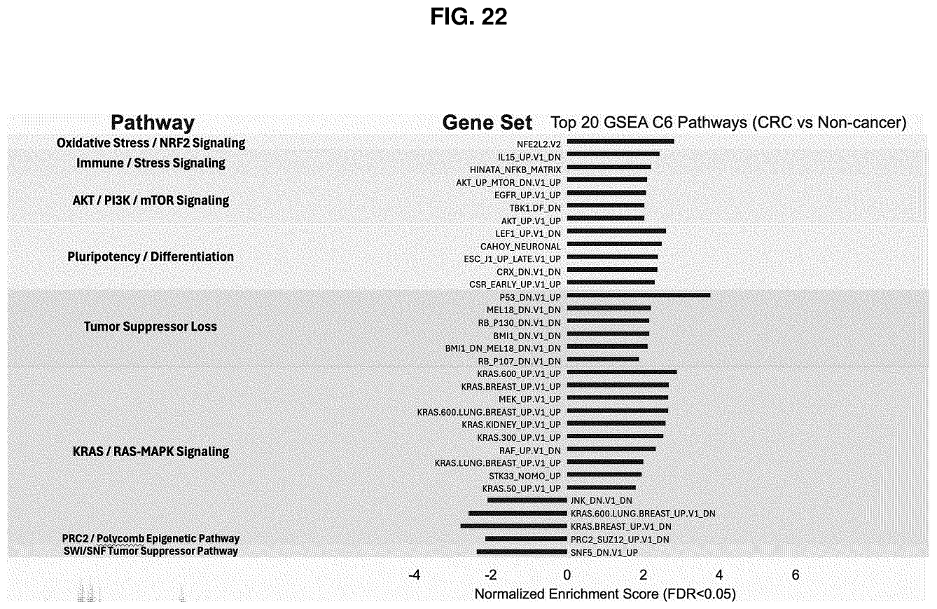

Analysis of MSigDB C6 oncogenic signatures further confirmed enrichment of oncogenic signaling programs, including activation of KRAS/RAS-MAPK signaling, concurrent tumor suppressor loss, increased pluripotency-associated programs, and activation of AKT/PI3K/mTOR signaling pathways. FIG. 22 is a bar plot showing the top 20 C6 gene sets with differential gene body 5hmC counts between CRC samples and non-cancer controls.

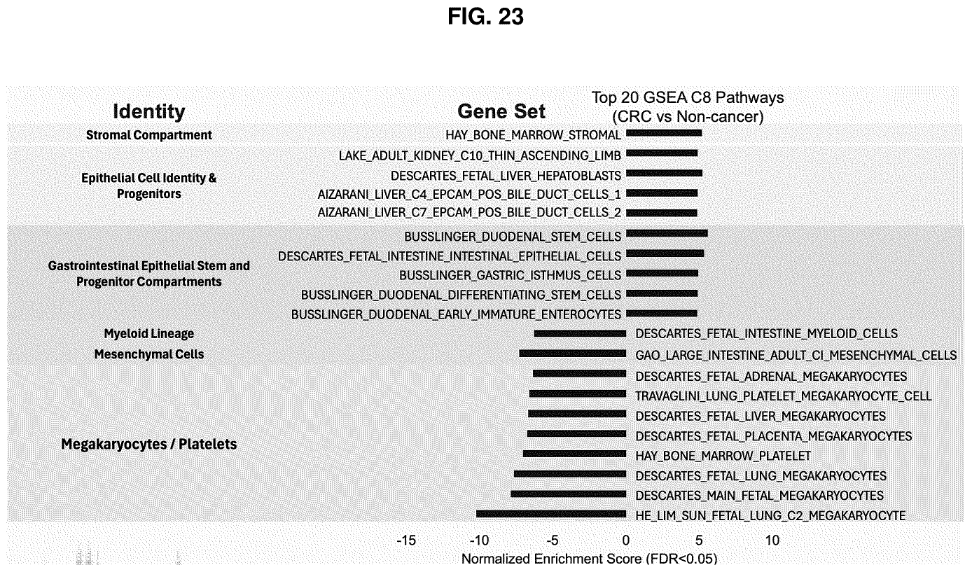

FIG. 23 is a bar plot showing the top 20 C8 gene sets with differential gene body 5hmC counts between CRC samples and non-cancer controls. NES>0 indicates differential 5hmC enrichment in CRC samples while NES<0 indicates enrichment in non-cancer controls (FDR<0.05).

Finally, examination of C8 cell type-specific gene sets demonstrated downregulation of megakaryocyte signatures, consistent with a reduced contribution of non-tumor-derived cfDNA. In contrast, enrichment of epithelial cell identity and progenitors, and stromal signatures suggests an increased proportion of tumor-derived cfDNA in CRC compared with non-cancer samples.

Overall, plasma cfDNA 5hmC profiling provided valuable insight into both pathway-level activation and shifts in contributing cell types, highlighting key biological differences between CRC and non-cancer states.

### Example 6

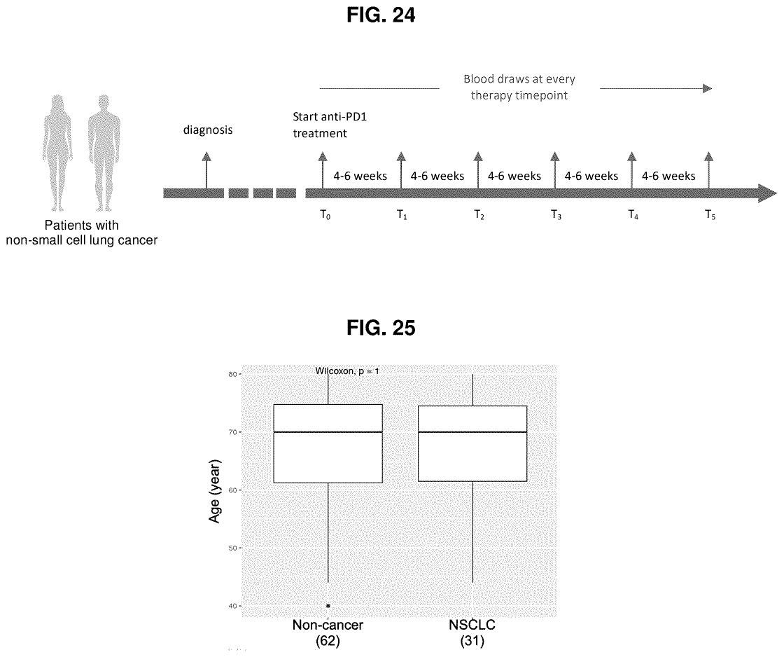

To assess the potential power of plasma 5hmC profiles in monitoring treatment response, NSCLC patients who received anti-PD-1 agents, pembrolizumab or nivolumab, as monotherapy, were examined. Blood was collected in cell-free DNA blood collection tubes (Streck) prior to starting anti-PD1 treatment and at 4-6 week intervals following treatment initiation (see FIG. 24). Response to therapy was evaluated by imaging and reported according to RECIST v1.1.

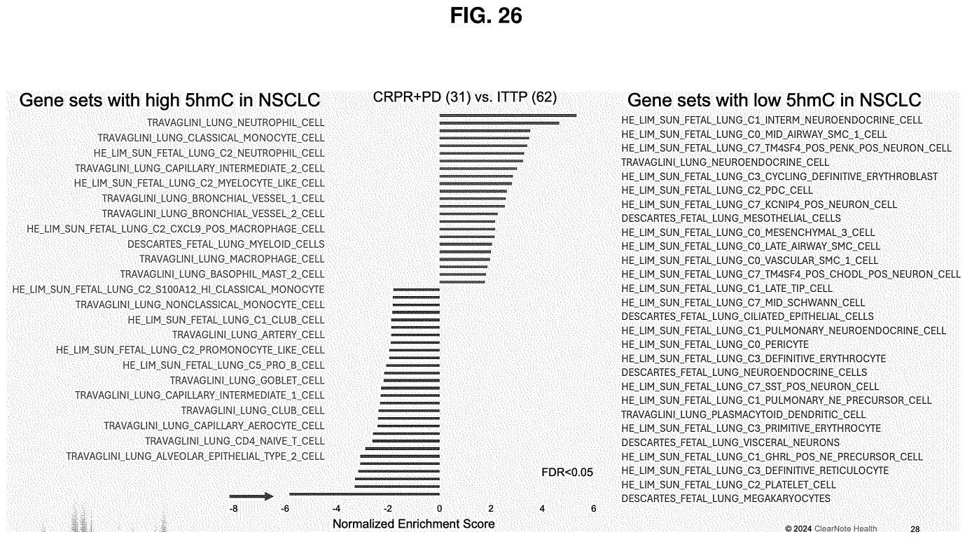

Response to therapy was evaluated by imaging and reported according to RECIST v1.1. All lung cancer samples (both responders and non-responders prior to treatment start) were combined and their characteristics were used to select non-cancer samples for age- and sex-matching. FIG. 25 shows the age distribution between NSCLC and non-cancer samples (Wilcoxon test), and the sex distribution of NSCLC and non-cancer samples was as follows: lung cancer (CRPR plus PD), 18 (58.1%) female, 13 (41.9%) male; non-cancer, 37 (59.7%) female, and 25 (40.3%) male. At baseline prior to treatment start, the genes with the lowest 5hmC levels in NSCLC patients were associated with lung megakaryocytes. See FIG. 26, which provides the results of GSEA using 5hmC counts over cell-type signature genes (C8) comparing NSCLC relative to non-cancer samples, where red (upper right) and blue (lower left) show elevated and reduced 5hmC representation in NSCLC relative to non-cancer, respectively.

Gene body 5hmC profiles were also examined at the time of response compared to anti-PD1 treatment start which revealed differential profiles for anti-PD-1 responders relative to non-responders after initiation of treatment. To determine whether 5hmC profiles in plasma-derived cfDNA can inform about the biological response to anti-PD-1 treatment, we performed gene set enrichment analysis using 5hmC counts over gene bodies. In responders, the top enriched pathways were heavily immune-related such as interferon-gamma response. Furthermore, 5hmC counts over genes in cell type signatures compared between time of response (T_{R}) and baseline (T₀) revealed several pathways associated with activation of immune response in responding patients and epithelial and stromal cell types in non-responders in a smaller set (FIG. 27) and a larger set (FIG. 28).

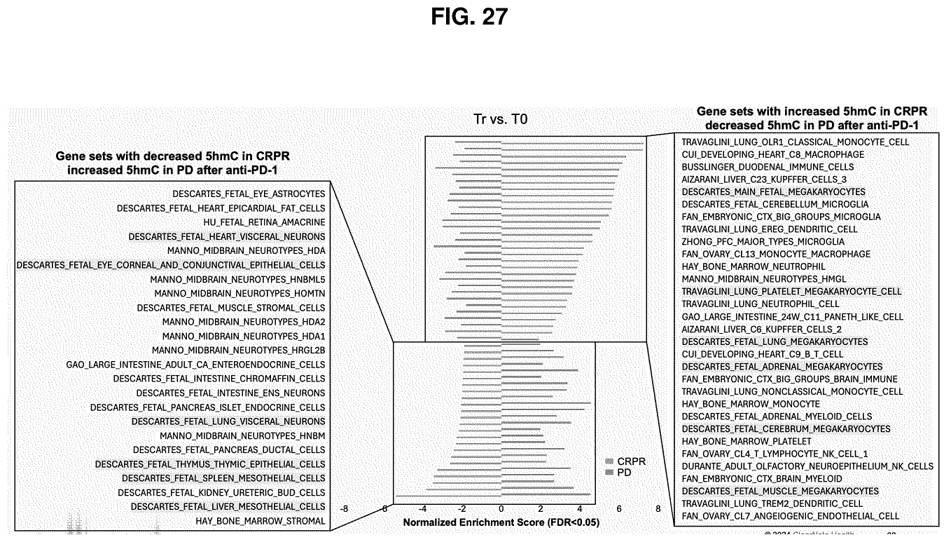

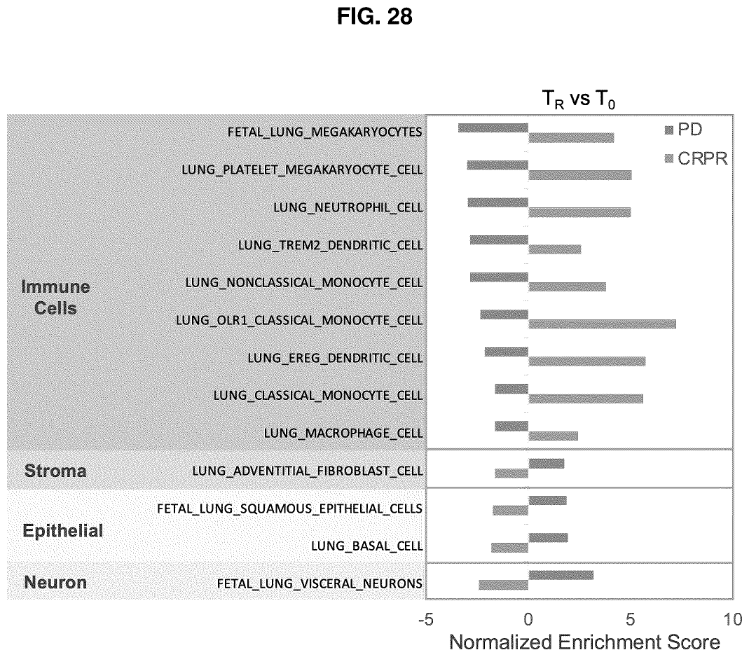

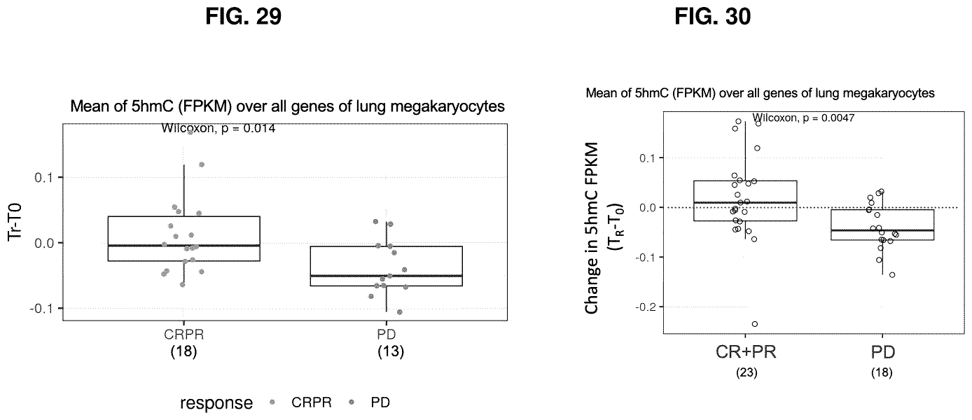

Immune genes with the highest differential 5hmC accumulation from time of radiologic response to baseline showed opposite behavior between responders (CR+PR) and non-responders (PD). Mean change of 5hmC levels over the top immune gene sets in response to anti-PD-1 treatment were significantly reduced in non-responders compared to responders (FIG. 29). The separation between responders and non-responders became even more significant with the increased sample size for both cohorts (FIG. 30).

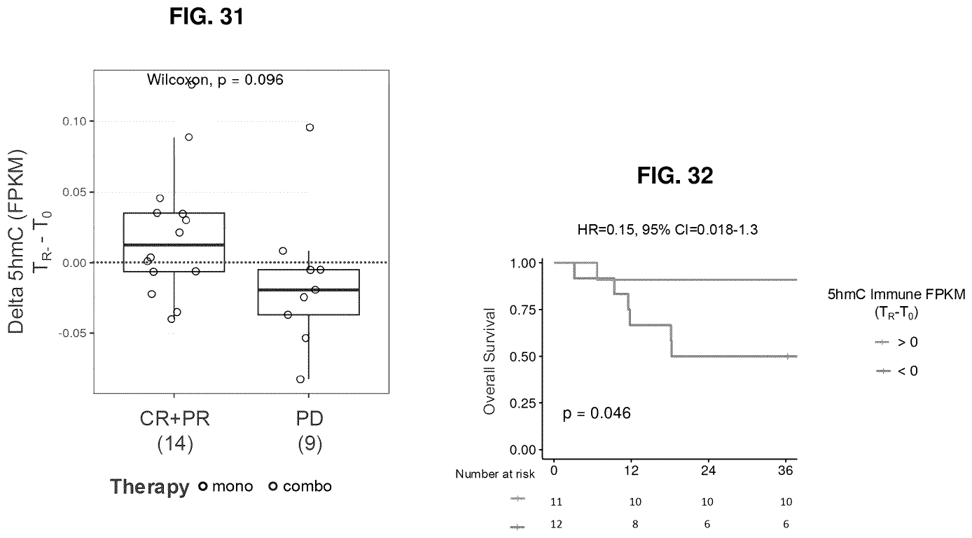

Lastly, we assessed whether a change in megakaryocyte-specific genes can impact patient outcome using subjects with at least a 3-year follow up. The median change in megakaryocyte 5hmC levels after immunotherapy was positive in responders (CR+PR) and negative in non-responders (PD), suggesting increased tumor load in non-responders (FIG. 31). Using the change in 5hmC levels over megakaryocyte genes, patients were stratified into positive (Tr−T0>0) and negative (Tr−T0<0) groups. Patients with increased 5hmC levels over megakaryocytes has significant better overall survival compared to patients with decreased 5hmC levels after treatment (FIG. 32).

### Example 7

Generation of Colorectal Cancer Detection Model Using Genic 5hmC Levels Profiled from Plasma Collected in Streck or EDTA Tubes:

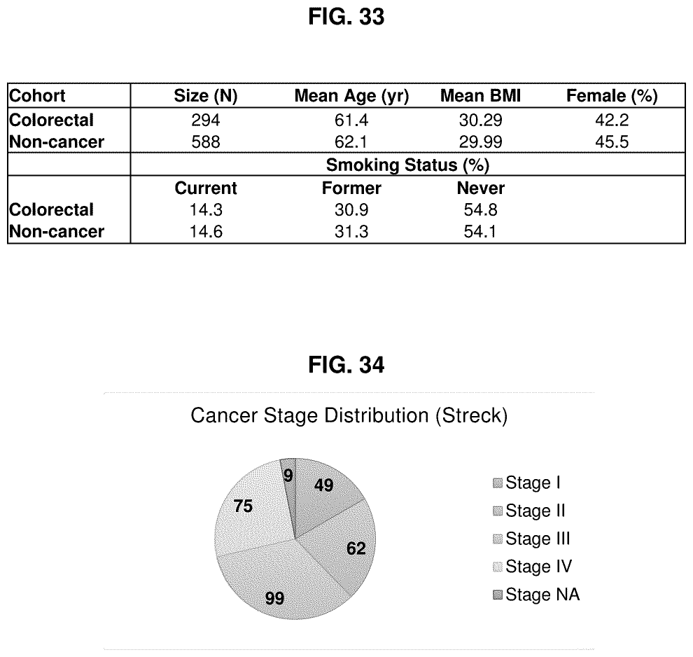

Plasma collected in Streck or EDTA tubes were used to isolate cfDNA which was then used to generate WGS and genome-wide 5hmC profiles. 5hmC levels over the gene bodies were utilized to generate a colorectal cancer detection model for each tube type. Age, BMI, sex and smoking status distributions were all balanced between the colorectal cancer cohort and the non-cancer cohort for Streck and EDTA tube-derived samples; see FIG. 33, which provides the clinical characteristics of the cancer and non-cancer cohorts; and FIG. 34, a pie chart indicating the cancer stage distribution of samples collected in Streck tubes. Approximately 54.8% of the CRC samples were late stage.

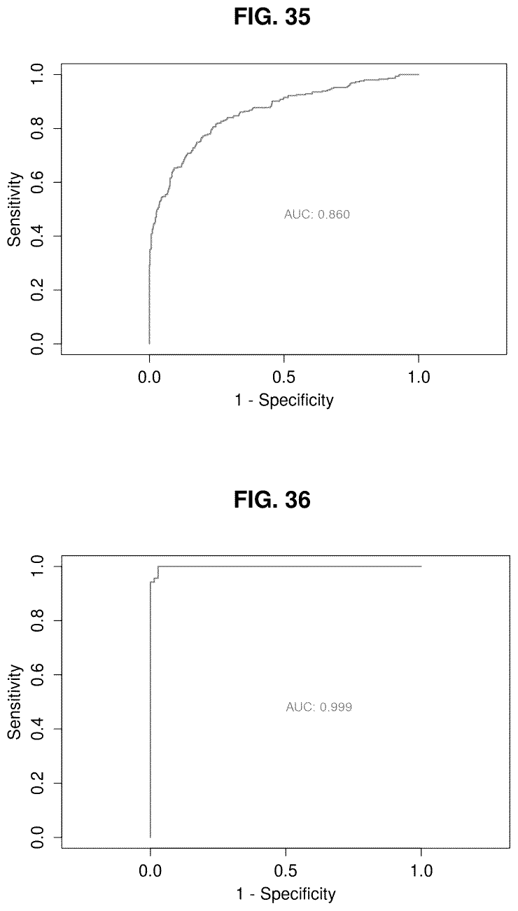

Using 5hmC levels over genes, a model was generated to detect colorectal cancer in plasma and 10-fold outer CV yielded an AUC of 0.86 with an overall outer CV sensitivity of 47.3% at 97.8% specificity. Sensitivity at 97.8% specificity was measured as 90.7% for stage IV samples with a 10-fold cross validation AUC of 0.964. FIG. 35 provides the receiving-operating characteristic (ROC) curve from predictive modeling using genic 5hmC on the training set with 294 CRC and 588 non-cancer cfDNA extracted from plasma collected in Streck tubes.

This model was tested on an independent data set comprised of 70 non-cancer (EDTA) and 75 CRC (EDTA) samples. This independent validation showed robust discrimination between colorectal cancer and non-cancer samples, with an area under the curve (AUC) of 0.939.

Similarly, a colorectal cancer detection model was generated using well-balanced CRC (n=69; 40.6% female) and non-cancer (n=70; 42.9% female) cohorts comprised of patients whose blood was collected in EDTA tubes. Remarkably, the ROC curve resulted in an AUC of 0.999 using 10-fold outer CV for the EDTA data set (FIG. 36).

### Example 8

Prognostic Value of Pre-Surgery Plasma-Derived Cfdna 5hmClevels Using Cancer Detection Score or Over Wnt/β-Catenin Pathway Genes: Elevated Scores Predict Shorter Overall Survival:

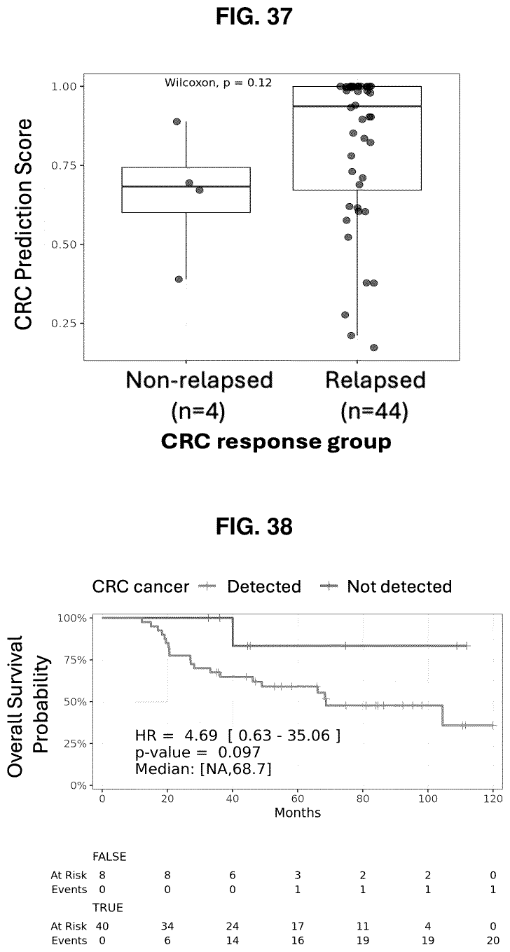

Two response groups, relapsed patients who showed progressive disease within 2 years post-surgery, and relapsed patients who remained disease-free after 2 years post-surgery, were scored with CRC cancer detection test. Overall, the patients with active disease (relapsed) cohort had a tendency to score higher than the group that remained disease-free (non-relapsed, FIG. 37).

All of these samples were then split into two groups based on their cancer scores: Cancer “detected” vs. “not detected.” Overall survival analyses based on the cancer test classification showed that patients classified as “cancer detected” had worse outcomes than those classified as “cancer not detected,” highlighting the potential prognostic value of plasma cfDNA 5hmC profiles (FIG. 38).

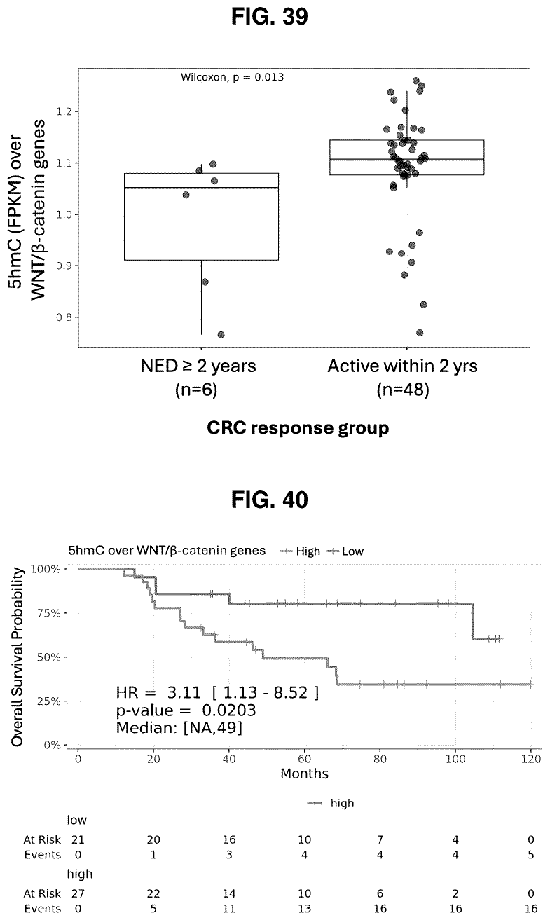

The Wnt/β-catenin signaling pathway has been linked to the initiation and progression of CRC (Bian et al., 2020, supra), and the overexpression of β-catenin in the nucleus has been associated with disease progression and worse prognosis for CRC patients (Chen et al., 2013; Wong et al., 2004; Cheah et al., 2002, cited supra). To test whether the prognostic methodology depicted by differential expression of the Wnt/β-catenin signaling pathway can be captured through plasma DNA, the overall 5hmC levels over Wnt/β-catenin signaling genes were compared between progressed patients and disease-free patients. As expected, progressive patients showed higher 5hmC relative to NEDs (FIG. 39), supporting literature findings.

The Wnt/β-catenin signaling pathway has been extensively implicated in the initiation and progression of colorectal cancer (CRC) (Bian et al., 2020), and nuclear overexpression of β-catenin has been associated with disease progression and poor prognosis in CRC patients (Chen et al., 2013; Wong et al., 2004; Cheah et al., 2002). To assess whether this prognostic signal can be captured using plasma-derived cfDNA, we compared overall 5hmC levels across Wnt/β-catenin signaling genes between patients with progressive disease and those with no evidence of disease (non-relapsed). Consistent with prior reports, patients with disease progression exhibited higher 5hmC levels relative to non-relapsed patients, supporting the ability of plasma cfDNA profiling to reflect clinically relevant Wnt/β-catenin pathway activity. Lastly, stratifying patients into “high” and “low” groups based on median Wnt/β-catenin 5hmC levels revealed that pre-surgery cfDNA 5hmC levels over WNT/β-catenin signaling pathway genes-whose overexpression has been associated with disease progression and poor prognosis in CRC patients-were significantly correlated with overall survival (HR=3.11, p=0.02) in stage IV CRC, further supporting the prognostic potential of plasma cfDNA 5hmC profiles in the surgical setting (FIG. 40).

### Example 9

### Correlation Between 5hmC Levels, Circulating Tumor DNA Fraction, and Chromatin Accessibility Over CRC-Specific Genes:

Griffin (Doebley et al., 2022) is a bioinformatics framework designed to analyze fragmentation and nucleosome protection patterns of cfDNA from plasma to extract chromatin accessibility information. Using low-pass whole-genome sequencing (LP-WGS) data from cfDNA, it profiles how cfDNA fragments align around genomic features like transcription factor binding sites (TFBSs) or chromatin accessibility regions. Griffin provides three readouts: mean coverage around the binding site, central coverage, and the amplitude. Because nucleosome protect DNA from degradation, the coverage patterns of cfDNA around these sites reflects chromatin structure and gene regulatory state: the lower the coverage, the higher the accessibility. Griffin output has also been shown to correlate well with circulating tumor DNA (ctDNA) fraction estimated by ichorCNA-another bioinformatics tool estimating ctDNA fraction using LP-WGS data derived from cfDNA by detecting copy number alterations (Adalsteinsson et al., 2017).

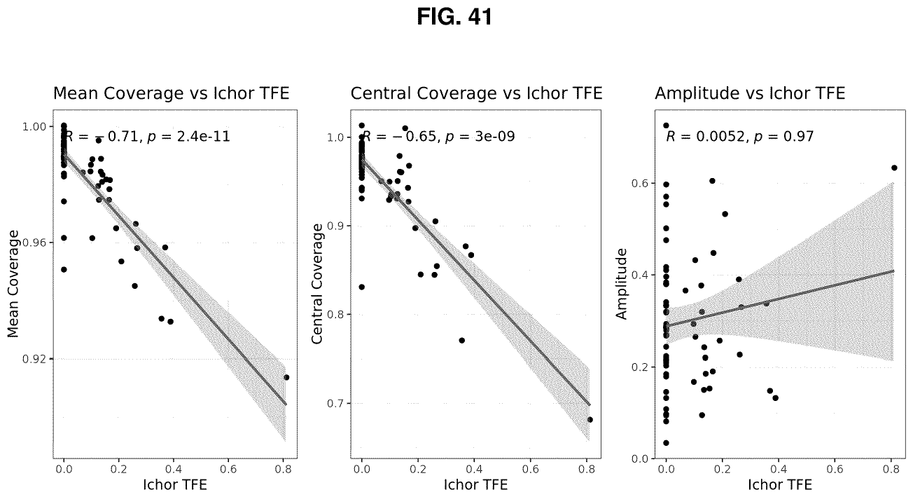

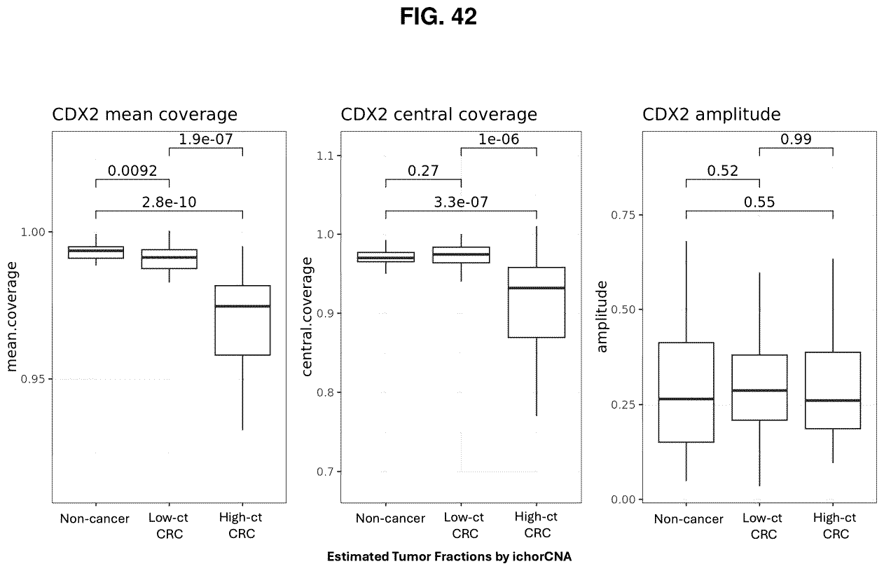

To evaluate whether gene body 5hmC levels correlate with tumor fraction and open chromatin accessibility, ichorCNA and Griffin tools were applied, respectively, to cfDNA-derived WGS data from 66 CRC (EDTA-derived) and 67 sex- and age-matched non-cancer controls. CDX2 (Caudal-type homeobox 2) is a lineage-defining transcription factor for intestinal epithelium and plays a central role in colorectal cancer (CRC) biology, diagnosis, and prognosis (Salari et al., 2012). As expected, CDX2 mean and central coverage was negatively correlated with the ichorCNA-estimated tumor fraction in CRC samples, indicating higher nucleosome accessibility (FIG. 41). Higher tumor fraction was correlated with higher nucleosome accessibility over CDX2 (FIG. 42).

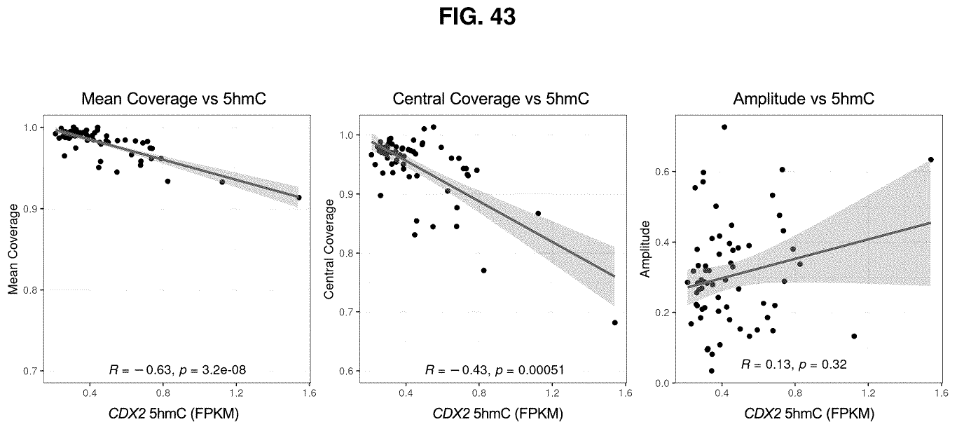

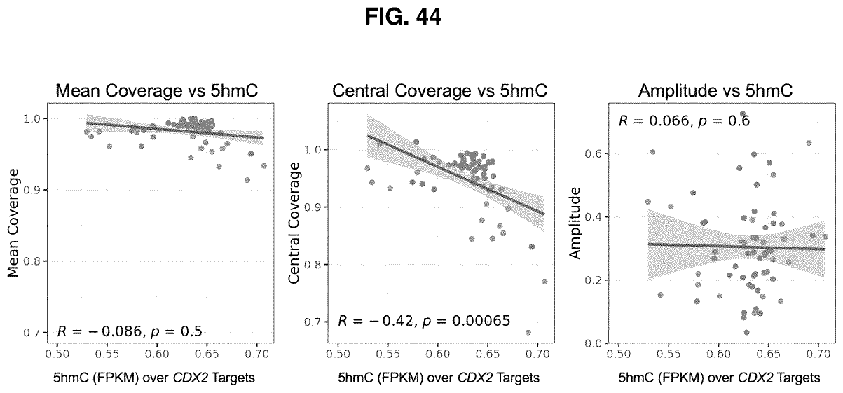

Similarly, a negative correlation was observed between CDX2 coverage as measured by mean/central coverage and 5hmC levels, indicating that higher CDX2 5hmC levels are associated with increased nucleosome accessibility (FIG. 43). This observation was further supported by analysis of 5hmC levels across CDX2 target regions, which showed a concordant increase in nucleosome accessibility and 5hmC, consistent with enhanced activation of the CDX2 transcriptional program. Overall, this integrative analysis revealed that increased nucleosome accessibility at the intestine-specific developmental regulator CDX2 was associated with enhanced epigenetic activation, as reflected by increased 5hmC at the CDX2 locus, and was likely driven by higher tumor content as estimated by ichorCNA. FIG. 44 presents scatter plots showing correlation between CDX2 mean coverage (left), central coverage (middle), and amplitude (right) shown against increased 5hmC levels over CDX2 target binding sites.

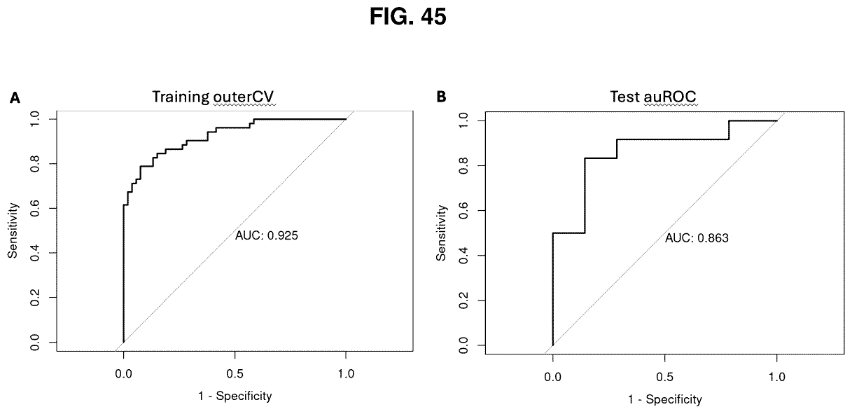

Lastly, Griffin's aggregated nucleosome accessibility measurements were made across 374 transcription factors to evaluate colorectal cancer (CRC) prediction performance using a GLMNET model. Mean coverage, central coverage, and amplitude across all 374 Griffin-provided transcription factors were used as features for model training. Eighty percent of CRC and non-cancer samples were used for training, with the remaining 20% reserved for independent testing. Model training was evaluated using 10-fold outer cross-validation, yielding an AUROC of 0.925 (FIG. 45, left). Evaluation on the held-out test set resulted in an AUC of 0.863 (FIG. 45, right), indicating strong predictive performance and the potential for enhanced CRC detection using cfDNA-derived nucleosome accessibility features.

### REFERENCES

Morris et al. (2022), “Treatment of Metastatic Colorectal Cancer: ASCO Guideline,” J. Clin. Oncol. 41:678-700.
Cavallo et al. (2021), “Solving the Conundrum of Young-Onset Colorectal Cancer: A Conversation with Kimmie Ng,” ASCO Post.
Nicholson et al. (2015), “Blood CEA levels for detecting recurrent colorectal cancer,” Cochrane Database Syst Rev. (12):CD011134.
Clark and Sanoff (2023), “Systemic Therapy for Metastatic Colorectal Cancer: General Principles”, in UpToDate, retrieved Apr. 15, 2024 from https://www.uptodate.com/contents/systemic-therapy-for-metastatic-colorectal-cancer-general-principles).
Chiorean et al. (2020), “Treatment of patients with late-stage colorectal cancer,” ASCO Resource-Stratified Guideline 6:414-438.
Bray et al. (2024), “Global cancer statistics 2022: GLOBOCAN estimates of incidence and mortality worldwide for 36 cancers in 185 countries,” CA: A Cancer Journal for Clinicians 74(3): 229-263.
Shields et al. (2021), “Immunotherapy for Advanced Non-Small Cell Lung Cancer: A Decade of Progress,” Am. Soc. Clin. Oncol. Educ. Book 41:1-23.
Moss et al. (2023), “Megakaryocyte- and erythroblast-specific cell-free DNA patterns in plasma and platelets reflect thrombopoiesis and erythropoiesis levels,” Nat Commun. 14(1): 7542.
Lefrançais et al. (2017), “The lung is a site of platelet biogenesis and a reservoir for haematopoietic progenitors,” Nature 544 (7648): 105-109.
Cunin and Nigrovic (2019), “Megakaryocytes as immune cells,” J Leukoc Biol. 105(6): 1111-1121.
Eisenhauer et al. (2009), “New response evaluation criteria in solid tumours: Revised RECIST guideline (version 1.1),” European J. Cancer 45(2): 228-247).
Spindler et al. (2023), “Circulating tumor DNA: Response Evaluation Criteria in Solid Tumors—can we RECIST? Focus on colorectal cancer,” Ther. Adv. Med Oncol. 15:15: 17588359231171580. doi: 10.1177/17588359231171580. PMID: 37152423; PMCID: PMC10154995.
Crutcher et al. (2020), “Biomarkers in the development of individualized treatment regimens for colorectal cancer,” Front. Med. (Lausanne) 9:1062423.
Mauri et al. (2022), “Liquid biopsies to monitor and direct cancer treatment in colorectal cancer,” Br J Cancer 127:394-407.
Malla et al. (2022), “Using Circulating Tumor DNA in Colorectal Cancer: Current and Evolving Practices,” J Clin Oncol. 40(24): 2846-2857.
Vidal et al. (2021), “Clinical Impact of Presurgery Circulating Tumor DNA after Total Neoadjuvant Treatment in Locally Advanced Rectal Cancer: A Biomarker Study from the GEMCAD 1402 Trial,” Clin Cancer Res. 27(10): 2890-2898.
Reinert et al. (2019), “Analysis of Plasma Cell-Free DNA by Ultradeep Sequencing in Patients With Stages I to III Colorectal Cancer,” JAMA Oncol. 5(8): 1124-1131.
Mann et al. (1947), “On a Test of Whether one of Two Random Variables is Stochastically Larger than the Other,” Annals of Mathematical Statistics 18(1): 50-60).
Casbon et al. (2011), “A method for counting PCR template molecules with application to next-generation sequencing,” Nucleic Acids Res. 39(12): 81.
Liu et al. (2019), “Bisulfite-free direct detection of 5-methylcytosine and 5-hydroxymethylcytosine at base resolution,” Nat Biotechnol 37:424-429.
Gallant (1990), “Perceptron-based learning algorithms,” IEEE Transactions on Neural Networks 1(2):179-91
Peng et al. (2005), “Feature selection based on mutual information: criteria of max-dependency, max-relevance, and min-redundancy,” IEEE Transactions on Pattern Analysis and Machine Intelligence 27(8):1226-38.
Cristianini and Shawe-Taylor (2000), “An Introduction to Support Vector Machines and other kernel-based learning methods,” Cambridge: Cambridge University Press.
Markey et al. (2002), “Perceptron error surface analysis; a case study in breast cancer diagnosis,” Comput Biol Med 32(2):99-109.
Cristescu et al. (2018), “Pan-tumor genomic biomarkers for PD-1 checkpoint blockade-based immunotherapy,” Science 362 (6411).
Thompson et al. (2021), “Serial Monitoring of Circulating Tumor DNA by Next Generation Gene Sequencing as a Biomarker of Response and Survival in Patients with Advanced NSCLC Receiving Pembrolizumab-Based Therapy,” JCO Precision Oncology 5:510-524.
Bindal et al. (2021), “Biomarkers of therapeutic response with immune checkpoint inhibitors,” Ann. Transl Med 9(12): 1040
Chen et al. (2021) Nature Portfolio 11:5040.
Song et al. (2011), “Selective chemical labeling reveals the genome-wide distribution of 5-hydroxymethylcytosine,” Nat. Biotechnol. 29(1): 68-72.
Han et al. (2016), “A highly sensitive and robust method for genome-wide 5hmC profiling of rare cell populations,” Mol. Cell 63:711-19.
Robinson et al. (2010, “edgeR: a Bioconductor package for differential expression analysis of digital gene expression data,” Bioinformatics 26:139-140.
Subramanian et al. (2005), “Gene set enrichment analysis: A knowledge-based approach for interpreting genome-wide expression profiles,” Proc Natl Acad Sci USA 102:15545-50.
Eisenhauer et al. (2009), “New response evaluation criteria in solid tumours: Revised RECIST guideline (version 1.1)” European J. Cancer 45:228-247.
Bian et al. (2020), “Transcriptional Regulation of Wnt/β-Catenin Pathway in Colorectal Cancer,” Cells 9(9):2125. https://doi.org/10.3390/cells9092125.
Chen et al. (2013), “β-catenin overexpression in the nucleus predicts progress disease and unfavourable survival in colorectal cancer: a meta-analysis,” PLOS One. 2013 May 24; 8(5):e63854. doi: 10.1371/journal.pone.0063854. PMID: 23717499; PMCID: PMC3663842.
Wong et al. (2004), “Prognostic and diagnostic significance of beta-catenin nuclear immunostaining in colorectal cancer,” Clin Cancer Res. 10(4): 1401-8. doi: 10.1158/1078-0432.ccr-0157-03. PMID: 14977843.
Cheah et al. (2002), “A survival-stratification model of human colorectal carcinomas with beta-catenin and p27kip1,” Cancer 95(12): 2479-86. doi: 10.1002/cncr.10986. PMID: 12467060.
Smith et al. (1996), “Overexpression of the c-myc proto-oncogene in colorectal carcinoma is associated with a reduced mortality that is abrogated by point mutation of the p53 tumor suppressor gene,” Clin Cancer Res. 2(6):1049-53. PMID: 9816266.
Toon et al. (2014), “Immunohistochemistry for myc predicts survival in colorectal cancer,” PLOS One. 2014 Feb. 4; 9(2):e87456. doi: 10.1371/journal.pone.0087456. PMID: 24503701; PMCID: PMC3913591.
Strippoli et al. (2020), “c-MYC Expression Is a Possible Keystone in the Colorectal Cancer Resistance to EGFR Inhibitors,” Cancers (Basel) 12(3):638. doi: 10.3390/cancers12030638. PMID: 32164324; PMCID: PMC7139615.
Lee et al. (2016), “Favorable prognosis in colorectal cancer patients with co-expression of c-MYC and β-catenin,” BMC Cancer 16:730. https://doi.org/10.1186/s12885-016-2770-7.
Wang, YN., Ruan, DY., Wang, Z X. et al. Targeting the cholesterol-RORα/γ axis inhibits colorectal cancer progression through degrading c-myc. Oncogene 41, 5266-5278 (2022). https://doi.org/10.1038/s41388-022-02515-3.
Ren, L., Meng, L., Gao, J. et al. PHB2 promotes colorectal cancer cell proliferation and tumorigenesis through NDUFS1-mediated oxidative phosphorylation. Cell Death Dis 14, 44 (2023). https://doi.org/10.1038/s41419-023-05575-9.
Wang et al. (2022), Cancers (Basel) 14(18): 4503. doi: 10.3390/cancers14184503. PMID: 36139663; PMCID: PMC9496738.
Kosinski et al. (2007), “Gene expression patterns of human colon tops and basal crypts and BMP antagonists as intestinal stem cell niche factors,” Proc. Natl. Acad. Sci. 104(39): 15418.
Doebley et al. (2022), “A framework for clinical cancer subtyping from nucleosome profiling of cell-free DNA,” Nat Commun. 13(1):7475. PMCID: PMC9719521.
Adalsteinsson et al. (2017), “Scalable whole-exome sequencing of cell-free DNA reveals high concordance with metastatic tumors,” Nat Commun. 8(1):1324. doi: 10.1038/s41467-017-00965-y. PMID: 29109393; PMCID: PMC5673918.
Salari et al. (2012), “CDX2 is an amplified lineage-survival oncogene in colorectal cancer,” Proc Natl Acad Sci USA 109(46):E3196-E3205. PMCID: PMC3503165.

1. A method for identifying differentially hydroxymethylated cytosine sites in cell-free DNA (cfDNA) useful as hydroxymethylation biomarkers in predicting a probability that a cancer patient will or will not experience recurrence of the cancer within a follow-up time period after treatment with a cancer therapy, wherein the method comprises:
(a) obtaining an initial cfDNA sample from each of a plurality of cancer patients prior to beginning treatment with the cancer therapy;
(b) determining a baseline count T₀ of hydroxymethylated cytosine sites in CPM at each of a plurality of candidate hydroxymethylation biomarker loci in the initial cfDNA samples;
(c) treating the patients with the colorectal cancer therapy;
(d) at the conclusion of the follow-up time period, confirming recurrence or nonrecurrence of cancer to identify a first population of cancer recurrent patients and a second population of cancer nonrecurrent patients;
(e) determining a correlation between the T₀ count at the hydroxymethylated cytosine sites and recurrence or nonrecurrence of cancer in the patients; and
(f) adopting as the hydroxymethylation biomarkers the hydroxymethylated cytosine sites exhibiting a sufficient correlation with recurrence or nonrecurrence.

2. A method for identifying differentially hydroxymethylated cytosine sites in cfDNA useful as hydroxymethylation biomarkers in predicting a probability that a cancer patient who has undergone treatment for the cancer will experience recurrence within a follow-up time period after the treatment, wherein the method comprises:
(a) obtaining an initial cfDNA sample from each of a plurality of cancer patients prior to beginning treatment with the cancer therapy;
(b) determining a baseline count T₀ of hydroxymethylated cytosine sites in CPM at each of a plurality of candidate hydroxymethylation biomarker loci in the initial cfDNA samples;
(c) treating the patients with the cancer therapy;
(d) at the conclusion of the follow-up time period, (i) confirming recurrence or nonrecurrence of cancer to identify a first population of cancer recurrent patients and a second population of cancer nonrecurrent patients and (ii) obtaining a subsequent cfDNA sample from each of the patients;
(e) determining a subsequent count T_{S} at each of the candidate hydroxymethylation biomarker loci in the subsequent cfDNA samples; and
(f) adopting as hydroxymethylation biomarker loci those candidate hydroxymethylation biomarker loci exhibiting a threshold p-value of less than 0.05 and a difference (of at least 1.5, wherein
$$ζ = ( {T}_{S} - {T}_{0} ) / {T}_{0} .$$

3. The method of claim 1, wherein the cancer is colorectal cancer.

4. The method of claim 3, wherein the colorectal cancer patient has metastatic colorectal cancer.

5. The method of claim 1, wherein the cancer is lung cancer.

6. The method of claim 5, wherein the cancer is non-small cell lung cancer.

7. The method of claim 1, wherein the cancer therapy comprises surgical resection of a cancerous lesion.

8. The method of claim 1, wherein the cancer therapy comprises radiation therapy.

9. The method of claim 1, wherein the cancer therapy comprises a chemotherapy.

10. The method of claim 1, wherein the cancer therapy comprises immunotherapy.

11. The method of claim 1, wherein the adopted hydroxymethylation biomarker loci are selected from at least one of the following gene sets: HALLMARK_WNT_BETA_CATENIN-SIGNALING; HALLMARK_MYC_TARGETSV1; HALLMARK_OXIDATIVE_PHOSPHORYLATION; and HALLMARK_G2M_CHECKPOINT.

12. A method for determining a probability that a cancer patient will or will not experience cancer recurrence within a follow-up time period after being treated with a cancer therapy, wherein the method comprises, prior to treatment:
(a) obtaining cfDNA sample from the patient, enriching for hydroxymethylated DNA in the sample, amplifying the hydroxymethylated DNA, and sequencing the amplified hydroxymethylated DNA in a manner that identifies 5hmC-containing fragments or sites in the DNA;
(b) determining a hydroxymethylation signature for the patient by identifying the extent of hydroxymethylation in the 5hmC-containing fragments or sites at each of the hydroxymethylation biomarker loci adopted according to the method of claim 1;
(c) using the 5hmC levels in the hydroxymethylation signature, calculating a probability score representing the probability that the cancer patient will not experience cancer recurrence within two years of treatment with the colorectal cancer therapy.

13. The method of claim 12, wherein the cancer patient has colorectal cancer.

14. The method of claim 13, wherein the cancer patient has metastatic colorectal cancer.

15. The method of claim 13, wherein the cancer therapy comprises surgical resection of a cancerous lesion.

16. The method of claim 13, wherein the cancer therapy comprises radiation therapy.

17. The method of claim 13, wherein the cancer therapy comprises a chemotherapy.

18. The method of claim 9, wherein the cancer therapy comprises immunotherapy.

19. The method of claim 12, wherein (c) comprises calculating the probability score using the hydroxymethylation signature and at least one additional feature type.

20. The method of claim 19, wherein the additional feature type is a clinical feature.

21. The method of claim 19, wherein the additional feature comprises at least one of cfDNA concentration in the cfDNA sample, 5hmC-containing fragment count in a selected genomic location, 5hmC-containing fragment size, copy number variation in the cfDNA, and methylation data.

22. A method for determining whether a cancer patient is responding to treatment with a cancer therapy, the method comprising:
(a) in a cfDNA sample obtained from the patient, determining a baseline count T₀ at each of the hydroxymethylation biomarker loci selected in claim 1;
(b) in a later cfDNA sample obtained from the patient, determining a later count T₀ at each of the hydroxymethylation biomarker loci selected in claim 1 during or subsequent to treatment with the therapy;
(c) calculating a value for
$${log}_{2} ( {T}_{Q} / {T}_{0} )$$
at each of the selected hydroxymethylated biomarker loci, and identifying the calculated values as xᵢ at each locus i or yⱼ at each locus j, wherein the xᵢ and yⱼ are positively and negatively correlated with treatment response, respectively;
(e) calculating a 5hmC molecular response score (MR_{5hmc}) for the patient using the equation
$${MR}_{5 hmC} = {μ}_{x} - {μ}_{y}$$
wherein μₓ is the mean of the xᵢ over i loci and μ_{y} is the mean of the yⱼ over j loci; and
(f) determining that the patient is responding to the therapy when the 5hmC molecular response score is positive.

23. The method of claim 22, wherein the cancer patient has colorectal cancer.

24. The method of claim 23, wherein the cancer patient has metastatic colorectal cancer.

25. The method of claim 23, wherein the cancer therapy comprises surgical resection of a cancerous lesion.

26. The method of claim 23, wherein the cancer therapy comprises radiation therapy.

27. The method of claim 23, wherein the cancer therapy comprises a chemotherapy.

28. The method of claim 23, wherein the cancer therapy comprises immunotherapy.

29. A method for determining that a likelihood that a patient has cancer is higher than a likelihood that the patient does not have cancer, comprising: obtaining a cfDNA sample from the patient; evaluating 5-hydroxymethylcytosine (5hmC) levels in megakaryocyte-derived cfDNA in the sample; comparing the megakaryocyte 5hmC levels with reference megakaryocyte-derived 5hmC levels observed for a healthy patient; and determining that the patient is more likely to have cancer based on the comparison if the patient megakaryocyte 5hmC levels are reduced relative to the reference megakaryocyte-derived 5hmC levels.

30. A method for determining a likelihood that a cancer patient will relapse within two years of undergoing a cancer treatment, comprising: obtaining a cfDNA sample from the patient; evaluating 5-hydroxymethylcytosine (5hmC) levels over Hallmark WNT/β-catenin pathway genes in the cfDNA sample; comparing the evaluated 5hmC levels with reference 5hmC levels over the Hallmark WNT/β-catenin pathway genes observed for reference cancer patients who do not exhibit recurrence within two years of undergoing the cancer treatment; determining that the patient is likely to exhibit recurrence within two years of undergoing the cancer treatment when the evaluated 5hmC levels are elevated relative to the reference 5hmC levels.

31. A method for determining a likelihood that a colorectal cancer patient will relapse within two years of undergoing a cancer treatment, comprising: obtaining a cfDNA sample from the patient; evaluating transcription factor 5-hydroxymethylcytosine (5hmC) levels in the patient cfDNA sample; comparing the evaluated transcription factor 5hmC levels with reference transcription factor 5hmC levels observed for reference cancer patients who do not exhibit recurrence within two years of undergoing the cancer treatment; and determining that the patient is likely to exhibit recurrence within two years of undergoing the cancer treatment when the evaluated 5hmC levels are elevated relative to the reference 5hmC levels.

32. The method of claim 31, wherein the transcription factor is CDX2.
